(19) United States
(12) Patent Application Publication
(10) Pub. No.: US 2024/0192231 A1
(43) Pub. Date: Jun. 13, 2024

(54) MARKERS FOR THE DIAGNOSIS OF LARGE VESSEL OCCLUSION

(71) Applicants: FUNDACIÓ HOSPITAL UNIVERSITARI VALL D'HEBRON - INSTITUT DE RECERCA, Barcelona (ES); SERVICIO ANDALUZ DE SALUD, Sevilla (ES); ABCDX SA, Genéve (CH)

(72) Inventor: Joan Montaner Villalonga, Barcelona (ES)

(21) Appl. No.: 18/555,141

(22) PCT Filed: Apr. 13, 2022
(86) PCT No.: PCT/EP2022/059866

§ 371 Date: Oct. 12, 2023

(30) Foreign Application Priority Data
Apr. 15, 2021 (EP) 21382322.2

(51) Int. Cl. G01N 33/68

(52) U.S. Cl. CPC G01N 33/6896; G01N 2800/2871; G01N 2800/52

(57) ABSTRACT

A method for diagnosing large vessel occlusion in a subject that is suffering from an ischemic stroke episode, the method including determining the level of certain proteins in an isolated sample of a subject. A method for differentiating ischemic stroke from any other condition selected from haemorrhagic stroke, transient ischemic attack and a stroke mimicking condition. Simplified kits including reagents to carry out the methods are also disclosed.

This application claims the benefit of European Patent Application EP21382322.2 filed the 15 Apr. 2021.

### TECHNICAL FIELD

The invention is related to the field of diagnostics or companion diagnostics, in particular to a method of diagnosing a large vessel occlusion during an ischemic stroke episode, and to the selection of proper therapies depending on the said diagnosis.

### BACKGROUND ART

Large vessel occlusion (LVO) is defined as the occlusion caused by blood clots that travel from elsewhere in the body and lodge within an artery in the brain. But sometimes, a large vessel occlusion can develop such a severely damaged inner lining, that a thrombus can form within the large vessel itself. In these fewer common instances, a large vessel stroke is a thrombotic stroke.

LVO is associated with unfavourable outcomes at 3 and 6 months in patients with acute ischemic stroke (AIS) (Gandhi C D, Al Mufti F, Singh iP, et al. Neuroendovascular management of emergent large vessel occlusion: update on the technical aspects and standards of practice by the Standards and Guidelines Committee of the Society of Neurointerventional Surgery. J Neurointerv Surg 2018; 10:315-20). Thus, an LVO is one of the more impairing causes when a subject has suffered from and survived an ischemic stroke episode.

Lakomkin et al found that 16 of the studies included in their systematic review used nine different definitions of LVO (different combinations of locations of arterial occlusions) and this might condition prevalence of LVO as shown by Waqas et al. (see Lakomkin N, Dhamoon M, Carroll K, et al. Prevalence of large vessel occlusion in patients presenting with acute ischemic stroke: a 10-year systematic review of the literature. J Neurointerv Surg 2019; 11:241-5; and Waqas M, et al. Effect of definition and methods on estimates of prevalence of large vessel occlusion in acute ischemic stroke: a systematic review and meta-analysis. J Neurointerv Surg. 2020 March; 12(3):260-265). A restrictive definition of LVO is the one disclosing the occlusion of any of the following arteries or arterial segments: occlusion of the intracranial carotid (ICA), basilar (BA), and M1 segment of middle cerebral artery occlusions (See. Vanacker P, Heldner M R, Amiguet M, et al.

Prediction of large vessel occlusions in acute stroke: National institute of Health Stroke Scale is hard to beat. Crit Care Med 2016; 44:e336-43). Although there is not a clear definition of LVO, nowadays it is still classified according to the localization of the occlusion, independently of its aetiology.

Recent trials have shown that endovascular treatment for LVO reduces morbidity and mortality for patients experiencing this form of severe acute ischemic stroke. Nevertheless, a minority of patients experiencing LVO receive endovascular treatment, often due to delays in reaching specialized hospitals where endovascular treatment can be performed (Rai A T et al. (2017). A population-based incidence of acute large vessel occlusions and thrombectomy eligible patients indicates significant potential for growth of endovascular stroke therapy in the USA. J Neurointerv Surg. 9:722-6). Patients experiencing acute stroke are often first encountered by Emergency Medical Services (EMS) professionals and early recognition of LVO stroke in the prehospital setting by EMS professionals can improve timely transport to endovascular centers and lead to better patient outcomes (Crowe R P, Myers J B, Fernandez A R, Bourn S, McMullan J T. The Cincinnati Prehospital Stroke Scale Compared to Stroke Severity Tools for Large Vessel Occlusion Stroke Prediction. Prehosp Emerg Care. 2020 Feb. 25:1-9.). Different Scales used for the diagnosis of LVO were compared by Crowe et al. (supra). They showed that in 2,415 patients that experienced an acute ischemic stroke, 26% of the patients with ischemic stroke (n=628) were diagnosed with LVO.

A Cincinnati Prehospital Stroke Scale (CPSS) score of 2 or higher demonstrated a sensitivity of 69% and a specificity of 78% for LVO. A Rapid Arteria occlusion evaluation (RACE) score of 4 or higher demonstrated a sensitivity of 63%, and a specificity of 73%. A Los Angeles Motor Scale (LAMS) score of 3 or higher demonstrated a sensitivity of 63%, a specificity of 72% and a positive VAN score demonstrated a sensitivity of 86%, and a specificity of 65%. Comparing the area under the ROC curve for each scale revealed no statistically significant differences in discriminative ability for LVO stroke. This makes evident the need of reliable markers of LVO.

In an attempt to provide reliable markers for the fast diagnosis of LVO, current inventors already proposed in the international patent application WO2020229691, using the level of circulating retinal binding protein-4 (RBP4) and of N-terminal fragment of B-type natriuretic peptide (NT-proBNP) in an isolated sample of a patient suffering from stroke. Accuracy of the diagnosis could be improved with the addition of the level of glial fibrillary acid protein (GFAP), and even by determining d-dimer in the isolated sample of the patient.

Despite all efforts, it is more than evident that there is a need in the art of improved or alternative tests using biomarkers to overcome the limitations of the methods disclosed in the art for the accurate diagnosis of LVO, to decide the best therapeutic approach for the patients and in the shortest time period. Moreover, meanwhile a clear definition of LVO is stablished, this need of reliable markers is more urgent, since it is a condition that requires a particular treatment (i.e., endovascular treatment or thrombectomy, which is not commonly available in all the centres of territory).

### SUMMARY OF INVENTION

Inventors have surprisingly found that by determining the levels of fatty acid binding protein (FABP), such as heart-type fatty acid binding protein (HFABP), alone or in combination with the value of at least one clinical parameter of the individuals, an accurate diagnosis of the suffering of an LVO in subjects with an episode of ischemic stroke can be provided. The method of diagnosis including the determination of the levels of FABP (e.g., HFABP) gives sensitivity values of the order of 40-60%, or higher, with accompanying high specificities of around 90-97%. Moreover, specificities of 100% for LVO can also be accomplished with accompanying sensitivities of above 50% for true positives LVO subjects, which in this field suppose high values in comparison with other markers or decision criteria.

Thus, the method provides the advantage of being able to detect the most of the LVO (sensitivity concept) with the less of false positives or the most of actual true negatives that are not LVO subjects (specificity concept).

Furthermore, the method of diagnosis is easily implementable by means of simplified kits that can be used at ambulatory level or at ambulances (i.e., as point of care (POC) devices), and this allows the fast determining of the marker, preferably within the two first hours after stroke symptoms onset. In addition, the fast determining of an actual LVO allows deciding timely transport to endovascular centres and lead to better patient outcomes.

Inventors provide an in vitro method for the diagnosis of LVO, comprising determining the level of one or more FABP members, in particular of HFABP, in an isolated sample of a subject.

Thus, a first aspect of the invention is an in vitro method for the diagnosis of LVO, comprising determining the level of a FABP member, such as HFABP, in an isolated sample of a subject, and comparing the said level with a reference value or range, wherein:
when the reference value or range (i.e., interval) is of a subject suffering from LVO and the level of a FABP, such as HFABP, in the isolated sample is equal or within said value or range, the subject is diagnosed of LVO; or
when the reference value is of a subject not suffering from LVO (i.e., of a health subject) and the level of FABP, such as HFABP, in the isolated sample is higher than the reference value (also termed herewith and in this case as a cut-off discriminating value), the subject is diagnosed of LVO.

The fatty-acid-binding proteins (FABPs) are a family of transport proteins for fatty acids and other lipophilic substances. These proteins are thought to facilitate the transfer of fatty acids between extra- and intracellular membranes. The family of FABPs include twelve (FABP 1-12) identified proteins and corresponding genes, which are mainly known by the tissue where they are mainly expressed. The most well characterized are FABPs 1 to 7. FABP1 is known as liver-type fatty acid-binding protein (LFABP) (human protein in Uniprot KB database with accession number P07148). FABP2 is known as Intestinal-type fatty acid-binding protein (IFABP) (human protein in Uniprot KB database with accession number P12104). FABP3 or heart-type fatty acid binding protein (HFABP), also known as mammary-derived growth inhibitor, is a protein that in humans is encoded by the FABP3 gene. Its Uniprot KB database accession number is P05413, with a protein sequence length of 133 amino acids that is processed into a mature form (version 4 of the sequence of Jan. 23, 2007; and version 199 of the database entry of Feb. 10, 2021). FABP4, also known as aP2 (adipocyte Protein 2) or as adipocyte-type fatty acid binding protein (AFABP) is a carrier protein for fatty acids that is primarily expressed in adipocytes and macrophages. (human protein in Uniprot KB database with accession number P15090). FABP5 is the known as epidermal-like fatty acid binding protein (EFABP) (human protein in Uniprot KB database with accession number Q01469). FABP6 or ileal (gastrotropin) (human protein in Uniprot KB database with accession number P51161). FABP7 or brain lipid binding protein (BLBP or B-FABP) is expressed in the brain (human protein in Uniprot KB database with accession number O15540).

It was precisely surprising that a member of FABP, such as HFABP, could provide such an accurate diagnosis method of LVO. HFBAP, for example, has been observed increased together with the levels of S100 calcium-binding protein B (S100B) in plasma in the acute phase of ischemic stroke in patients with large artery atherosclerosis (LAA) and cardioembolism (CE), in relation with patients of small vessel disease type but without statistical significance (see So-Young et al. (2012). Plasma heart type fatty acid binding protein level in acute ischemic stroke: comparative analysis with plasma S100B level for diagnosis of stroke and prediction of long-term clinical outcome. Clinical Neurology and Neurosurgery 115, pp.: 405-410). However, these diseases (i.e., LAA, CE or SVD are different events that an LVO, and, in any case a non-significant correlation was observed for HFABP. In the international patent application WO2018229144, HFABP (i.e., FABP3) is proposed in combination with other markers for the distinction of ischemic stroke (IS) and transient ischemic attack (TIA) from a haemorrhagic stroke (HS) (see data in Table 3 of WO2018229144). This document is silent about LVO diagnosis, or of selecting candidates to reperfusion therapies).

Examples below show determination of HFABP among the members of the FABP family since a kit was available for determining that protein even at ambulance level. Nonetheless, other FABP, in particular BFABP, could be determined with the appropriate kits or reagents for the purpose of carrying out the methods of the invention.

The invention also provides, as a second aspect, an in vitro method for selecting a subject for a reperfusion therapy, said subject suffering LVO in the course of an ischemic stroke episode, the method comprising determining the level of a fatty acid binding protein (FABP), in particular of heart-type fatty acid binding protein (HFABP), in an isolated sample of the subject, and then including the step of comparing the said level with a reference value or range, wherein:
when the reference value or range (interval) is of a subject suffering from LVO and the level of FABP, in particular HFABP, in the isolated sample is equal or within said value or range, the subject is diagnosed of LVO and candidate to reperfusion therapy; or
when the reference value or range is of a subject not suffering from LVO and the level of FABP, in particular of HFABP, in the isolated sample is higher than the reference value or cut-off discriminating value, the subject is diagnosed of LVO and candidate to reperfusion therapy.

Thus, this method is encompassed as a companion diagnostic method.

Since FABP, in particular HFABP, and other biomarkers identified in the present invention allow diagnosing LVO and considering that, then particular therapies are applied to these subjects, the invention also provides the above-mentioned method of selecting a subject for a reperfusion therapy, in particular for a thrombectomy. Thus, both aspects are intimately and conceptually related.

Indeed, the main goal of a fast diagnosis of LVO, ideally at ambulance level, allows deciding the best centre in the proximity where the subject will be able to receive the adequate treatment. LVO is treated in these specialized centres by means of a thrombectomy, which is a type of reperfusion therapy. In other words, the methods of the previous aspects allow shifting the patient to the right hospital gaining time to start the right therapy.

Reperfusion therapies are disclosed in more detail below, but they include those interventions allowing to restore blood flow, either through or around, blocked arteries. Reperfusion therapy can be performed by means of drugs, such as thrombolytics (antithrombotic agents) and fibrinolytics. Reperfusion therapy can also be performed endovascular procedures, such as a thrombectomy. LVO diagnosed subjects are always treated by a thrombectomy. However, with the use of reference cut-offs allowing a 100% specificity for an actual LVO, the subject can also be selected for thrombolytics meanwhile the thrombectomy is finally performed. As indicated and unexpectedly, with the determining of the levels of a FABP member family, in particular HFABP, high sensitivity values are achieved even with a 100% of specificity for this condition (i.e., LVO). Thus, with the determining of the levels of a FABP, in particular of HFABP levels at ambulance or at ambulatory, a fast decision for a right provisional therapy can be taken.

In the same way, with cut-off references of HFABP as an example of FABP family member allowing 100% of specificity for an LVO, even the subject could be selected for a fast therapy including neuroprotective drugs meanwhile the subject is being prepared for the resolutive thrombectomy.

Indeed, another aspect of the invention is an in vitro method for selecting a subject to be shifted to a centre for thrombectomy and/or for a direct transfer to an angio-suite (DTAS) in the said centre for thrombectomy, the method comprising determining the level of a fatty acid binding protein (FABP) in an isolated sample of the subject, and comparing the said level with a reference value or range, wherein:
(a) the subject is selected to be shifted to a centre for thrombectomy when the level of FABP in the isolated sample is equal to a reference value or within a range of a subject suffering from LVO; or
(b) the subject is selected to be shifted to a centre for thrombectomy when the level of FABP in the isolated sample is higher than a reference value or range of a subject not suffering from LVO; and/or
(c) the subject is selected for a direct transfer to an angio-suite in the said centre for thrombectomy, when the level of FABP in the isolated sample is higher than a reference value that allows discriminating with a specificity of 100% between a subject suffering from either an LVO or a haemorrhagic stroke from a subject suffering from either a non-LVO ischemic or a mimic stroke (i.e. a stroke mimicking condition).

Another aspect of the invention is a kit comprising reagent means for detecting simultaneously a FABP family member, and of one or more of the levels of BNP, DDi, and GFAP.

Yet another aspect of the invention is the use of a kit as defined above for the diagnosis of LVO in a subject, or for selecting a patient suffering from ischemic stroke for a reperfusion therapy, in particular for a thrombectomy.

In yet another aspect, the invention aims also the use of means for detecting the presence of any of HFABP, or of any other FABP member of the family in a test sample, said means being selected from the group consisting of immunoassays, protein migration, chromatography, mass spectrometry, turbidimetry, nephelometry and polymerase chain reaction (PCR), for carrying out the method for diagnosing LVO, as defined in the first aspect; or for selecting a patient suffering from stroke for a reperfusion therapy, mainly for a thrombectomy treatment.

Another aspect of the invention is an in vitro method for the selection of a subject for a reperfusion therapy and/or for a therapy with neuroprotective drugs, the method comprising:
(a) determining the level of a FABP, in particular HFABP, in an isolated sample of the subject, in combination with:
   one or more clinical parameters selected from the group consisting of blood pressure, including systolic blood pressure (BSP) and/or diastolic blood pressure (DBP) and/or mean blood pressure (mean BP), glycemia, age, scores from systematic assessment tools of stroke-related neurologic deficits, time from onset of symptoms, and gender; and
   the level of one or more of the following proteins: a natriuretic peptide, in particular selected from BNP and ANP; D-dimer (DDi); and GFAP in the isolated sample of the subject; and
(b) the step of comparing the levels of FABP, in particular of HFABP, and of the one or more of the clinical parameters and of the levels of the other proteins, with a corresponding reference value or interval of a subject suffering from ischemic stroke and/or from a subject suffering from a condition selected from an haemorrhagic stroke, a transient ischemic attack or a stroke mimicking condition; and wherein the subject is diagnosed as suffering from an ischemic stroke and as candidate to a reperfusion therapy and/or candidate for a therapy with neuroprotective drugs, when the level of FABP, in particular of HFABP, and also of the one or more of the other determined proteins and/or the values of the one or more of the determined clinical parameters are within the value or interval of values from a subject suffering from ischemic stroke.

Also, another aspect of the invention is an in vitro method for the prognosis of a patient suffering LVO, comprising determining the level of FABP in an isolated sample of said patient, and comparing the said level with a cut-off value stratifying the patients according to either the dependency degree and/or the mortality rate, wherein if the level of FABP in the sample is higher than the cut-off value, the subject suffering LVO is also classified as:
(i) having a prognosis defined by a dependency degree greater than 2 according to modified ranking score (mRS), and determined within 1-5 months after stroke onset; and/or
(ii) having a prognosis defined by a three-month after onset mortality rate comprised from 25%-35%.

This prognostic method is of high interest, since it will aid in future decisions about personalizing endovascular therapies and to add neuroprotective strategies in those with predicted poor outcome in spite of successful reperfusion.

Yet another aspect of the invention is a computer-implemented method for carrying out the methods as defined in any of the first and second aspects, in which after the determination of the level of a FABP, in particular HFABP, said level is given a value and/or a score, and optionally it is computed in a mathematical formula to obtain a computed value; wherein in function of the said level, score and/or computed value, a decision is taken between the options of suffering an LVO and/or being candidate to a reperfusion therapy.

### DETAILED DESCRIPTION OF THE INVENTION

All terms as used herein in this application, unless otherwise stated, shall be understood in their ordinary meaning as known in the art. Other more specific definitions for certain terms as used in the present application are as set forth below and are intended to apply uniformly through-out the specification and claims unless an otherwise expressly set out definition provides a broader definition.

As used herein, the indefinite articles “a” and “an” are synonymous with “at least one” or “one or more.” Unless indicated otherwise, definite articles used herein, such as “the” also include the plural of the noun.

The term “patient” (or subject), as used herein, refers to any subject which show one or more signs or symptoms typically associated with stroke such as sudden-onset face weakness, arm drift, abnormal speech as well as combination thereof such as the FAST (face, arm, speech, and time), hemiplegia and muscle weakness of the face, numbness, reduction in sensory or vibratory sensation, initial flaccidity (hypotonicity), replaced by spasticity (hypertonicity), hyperreflexia, obligatory synergies and, in particular, when they appear in one side of the body (unilateral), altered smell, taste, hearing, or vision (total or partial), drooping of eyelid (ptosis) and weakness of ocular muscles, decreased reflexes (e.g. gag, swallow, pupil reactivity to light), decreased sensation and muscle weakness of the face, balance problems and nystagmus, altered breathing and heart rate, weakness in sternocleidomastoideole with inability to turn head to one side, weakness in tongue (inability to protrude and/or move from side to side), aphasia, dysarthria, apraxia, visual field defect, memory deficits, hemineglect, disorganized thinking, confusion, hypersexual gestures, lack of insight of his or her, usually stroke-related, disability, altered walking gait, altered movement coordination, vertigo, headache and or disequilibrium. The term “patient”, as used herein, refers also to all animals classified as mammals and includes, but is not restricted to, domestic and farm animals, primates and humans, e.g., human beings, non-human primates, cows, horses, pigs, sheep, goats, dogs, cats, or rodents. Preferably, the patient is a male or female human of any age or race. Preferably the patient suffers stroke.

The term “selecting a patient for a therapy”, in this particular description for a “thrombectomy” or “endovascular treatment” (used as synonymous), optionally in combination with other reperfusion techniques, such as the administration of thrombolytics and fibrinolytics, relates to the identification of a patient for a therapy designed to cure a disease or palliate the symptoms associated with one or more diseases or conditions. In the particular case of a therapy for an LVO in a patient suffering stroke, it is understood any therapy which abolishes, retards or reduces the symptoms associated with stroke and, more in particular, with ischemic stroke, due to the removing of the thrombus occluding the large vessel. Indeed, the patient is selected for a “reperfusion therapy”, which relates to a medical treatment to restore blood flow, either through or around, blocked arteries. Reperfusion therapy includes drugs and endovascular procedures. The drugs are thrombolytics (antithrombotic agents) and fibrinolytics used in a process called thrombolysis. Interventions performed may be minimally-invasive endovascular procedures for removing the thrombus (thrombectomy), with the possible use of one or more stent-retrievers, aspiration techniques or alternatives devices that combine both stent-retrievers and aspiration. Other surgeries performed are the more invasive bypass surgeries that graft arteries around blockages. “Mechanical thrombectomy”, or simply thrombectomy, is the interventional procedure of removing a blood clot (thrombus) from a blood vessel. It is commonly performed in the coronary arteries (interventional cardiology), peripheral arteries (interventional radiology) and cerebral arteries (interventional neuroradiology), the later case the one referred to in this description.

The selection or diagnosis of a patient, although preferred to be, need not be adequate for 100% of the subjects selected according to this first method of the invention. The term, however, requires that a statistically significant portion of subjects be correctly selected. Whether the selection or diagnosis of a patient in a population of subjects is statistically significant can be determined by the person skilled in the art using various well known statistic evaluation tools, e.g., determination of confidence intervals, p-value determination, Student's t-test, Mann-Whitney test, etc. Details are found in Dowdy and Wearden, Statistics for Research, John Wiley & Sons, New York 1983. Preferred confidence intervals are at least 50%, at least 60%>, at least 70%>, at least 80%>, at least 90%>, or at least 95%. The p-values are, preferably, 0.01, 0.05, 0.005, 0.001 or lower. In the next section defining the term “reference value or cut-off” or “reference range/interval”, the criteria for particularly desired specificities in this field and the impact in the sensitivity of the methods is disclosed in more detail.

The term “ischemic stroke” (abbreviated IS) refers to the physical blockage of blood flow to an area of the brain, causing brain cells in the area to die. Ischemic strokes can further be divided into thrombotic and embolic strokes. Thrombotic strokes occur when a brain artery is blocked by a blood clot formed in the brain. Embolic strokes are caused by a thrombus, which is formed in a peripheral artery or in the heart that travels to the brain where it produces ischemia. When due to an embolic or a thrombotic stroke a large vessel (artery) in the brain is occluded, this is also defined as an ischemic stroke with large vessel occlusion (abbreviated as LVO, or LVO IS in this description). In the opposed, a non-LVO IS includes any ischemic stroke type without a large vessel occlusion. Another type of ischemic strokes are lacunar strokes due to the occlusion of a small cerebral artery (i.e., these are a type that can also be classified as a non-LVO IS). On the contrary, an “hemorrhagic stroke” (abbreviated ICH if intracerebral haemorrhage), as used herein, refers to a bleeding into the brain tissue due to a blood vessel burst. A stroke mimic, or stroke mimicking condition, is defined as a disease or condition that presents with a stroke-like clinical picture but without neurologic tissue infarction. Several clinical syndromes can present with symptoms or signs that resemble an acute ischemic stroke and, thus, differentiation between a stroke and a stroke mimic is difficult due to the wide variety of overlapping clinical presentations. This is a real challenge for physicians, because of the potential adverse effects of interventional stroke therapies. Few are nowadays the markers in isolated samples of patients that allow distinguishing actual strokes from mimics.

The term “level of expression of one or more proteins” or “level of one or more proteins in a sample” relates to the amount of the protein expressed as a concentration, usually the weight of the protein in a volume of sample.

A “device” or “kit” in the sense of the invention is an assay or method to determine a (combination of) biomarkers (levels of proteins of interest in a sample) or panel of biomarkers according to the invention that can be used to perform an assay or method for the diagnosis of LVO or for the selection of a patient for a thrombectomy or any other reperfusion therapy. Examples are carrier plates, test stripes, biochip arrays, or the like known in the art including the reagent means to detecting the presence and level of the proteins of interest. The kits, as used herein, refer to a product containing the different reagents (or reagent means) necessary for carrying out the methods of the invention packed so as to allow their transport and storage. Materials suitable for packing the components of the kit include crystal, plastic (e.g. polyethylene, polypropylene, polycarbonate), bottles, vials, paper, or envelopes. Instructions in different formats for carrying out the method are, in some embodiments also included in the said kits. Particular formats of the instructions are selected from leaflets, electronic supports capable of storing instructions susceptible of being read or understood, such as, for example, electronic storage media (e.g. magnetic disks, tapes), or optical media (e.g. CD-ROM, DVD), or audio materials.

“Marker” or “biomarker” or “molecular marker” or “molecular biomarker” or “protein level” or “gene expression level” (all used interchangeable) in the sense of the invention, is any useful biomarker to detect in a sample of preferably blood, plasma, saliva, tears, CSF or urine, the presence of a LVO in the subject. The markers (i.e., protein levels) are used in a suitable assay setup wherein in particular the specificity is set to 90-100% and the sensitivity is in particular more than 40%.

A marker “panel” in the sense of the invention is a combination of at least two biomarkers (the levels of two proteins in the sample), in particular two or three or four markers, used in combination in a suitable setup or device, an optionally used in combination with other clinical parameters or inherent features of the subject.

As previously indicated, the invention relates in a first aspect to an in vitro method for the diagnosis of large vessel occlusion (LVO), comprising determining the level of a FABP, in particular, heart-type fatty acid binding protein (HFABP) in an isolated sample of a subject.

Large vessel occlusion (LVO) is defined, as indicated, as the occlusion caused by blood clots that travel from elsewhere in the body and lodge within an artery in the brain. Thus, it is properly a large brain vessel occlusion.

The invention also relates, in a second aspect, to the direct practical application of the previous one, thus to an in vitro method for selecting a subject for a reperfusion therapy, said subject suffering LVO in the course of an ischemic stroke episode, the method comprising determining the level of heart-type fatty acid binding protein (HFABP) in an isolated sample of the subject.

In a particular embodiment of the in vitro method for selecting a subject for a reperfusion therapy of the second aspect, the reperfusion therapy is a thrombectomy; or a thrombectomy in combination with a previous administration of a thrombolytic agent and/or fibrinolytic agent and/or a neuroprotective drug.

In a particular embodiment of the in vitro method for selecting a subject for a reperfusion therapy of the second aspect, the reperfusion therapy is a thrombectomy.

Therefore, in a more particular mode, the second aspect of the invention is an in vitro method for selecting a subject for a thrombectomy that comprises the above steps of first determining the level of a fatty acid binding protein (FABP), in particular heart-type fatty acid binding protein (HFABP), in an isolated sample of the subject, and second including the step of comparing the said level with a reference value or range, wherein:
when the reference value or range (interval) is of a subject suffering from LVO and the level of FABP, such as HFABP, in the isolated sample is equal or within said value or range, the subject is diagnosed of LVO and candidate to the thrombectomy; or
when the reference value or range is of a subject not suffering from LVO and the level of FABP, such as HFABP, in the isolated sample is higher than the reference value or cut-off discriminating value, the subject is diagnosed of LVO and candidate to the thrombectomy.

As will be illustrated in the examples and with the data retrieved from the inventors, the method allows the implementation of a novel strategy of selecting the patients to be directly derived to the thrombectomy centre, or even directly to the angio-suite where thrombectomy is performed (direct shift). This novel strategy is directly correlated with a better prognosis of the patients in terms of dependency degree after the stroke episode. Indeed, the patient can be adequately recommended for a thrombectomy two hours before it had been done with other protocols (i.e., first deriving the patient to a closest centre for stroke treatment but not capable for thrombectomy). The patient can be adequately recommended for a thrombectomy even two hours and a half, before it had been done with other protocols, if the subject is shifted to angio-suite directly.

In a particular embodiment of any of the in vitro method for the diagnosis of large vessel occlusion (LVO), or the in vitro method for selecting a subject for a reperfusion therapy, LVO is the one disclosing the occlusion of one or more of following arteries or arterial segments: occlusion of the intracranial carotid (ICA), basilar (BA), and M1 segment of middle cerebral artery occlusions.

In also another particular embodiment of any of the in vitro method for the diagnosis of large vessel occlusion (LVO), or the in vitro method for selecting a subject for a reperfusion therapy, the FABP is selected from one or more of liver-type fatty acid-binding protein (LFABP), Intestinal-type fatty acid-binding protein (IFABP), heart-type fatty acid binding protein (HFABP), adipocyte-type fatty acid binding protein (AFABP), epidermal-like fatty acid binding protein (EFABP), FABP6 (or ileal or gastrotropin), and brain lipid binding protein (BLBP or B-FABP)

In a more particular embodiment FABP is selected from HFABP and BFABP, and combinations thereof. Even more in particular FABP is HFABP.

In a particular embodiment of any of the in vitro method for the diagnosis of large vessel occlusion (LVO), or the in vitro method for selecting a subject for a reperfusion therapy, the method further comprised determining one or more clinical parameters.

The term “clinical parameters” or clinical data, as used herein, refers to person demographics (age or date of birth, race and/or ethnicity), patient clinical symptoms or signs related to stroke related diseases/conditions. The term also includes laboratory parameters, such as the determination of glycemia. These clinical parameters are annotated values regarding the above features, and they are considered in the decision of the diagnosis or selection for a therapy in combination with the levels of a FABP, such as HFABP in the isolated sample of a subject. These clinical parameters are taken or retrieved from the subject, as usual parameters or information considered when a suffering of stroke is suspected, even any parameter that can be measured or determined at ambulance level (i.e., electrocardiogram, examination of the ocular fundus, etc.). These annotated values of the clinical parameters are, in particular embodiments, computed together in a mathematical formula or algorithm with the detected levels of a FABP, such as HFABP and, if determined, also with the levels of other proteins, of a subject suspected from suffering a stroke. The output of the said computation or of the applied algorithm will provide information for the taking of a decision (i.e., classification for a therapy and/or diagnosis of a condition such as an LVO, for example).

In a more particular embodiment, the clinical parameters of the subject are selected from the group consisting of blood pressure, including systolic blood pressure (SBP) and/or diastolic blood pressure (DBP), mean blood pressure (mean BP), glycemia, age, scores from systematic assessment tools of stroke-related neurologic deficits, time from onset of symptoms, gender, presence of auricular fibrillation, and combinations thereof. In even a more particular embodiment the clinical parameters are selected from scores from systematic assessment tools of stroke-related neurologic deficits; blood pressure, including systolic blood pressure (SBP) and/or diastolic blood pressure (DBP), mean BP; and combinations thereof.

The term “systematic assessment tool stroke-related neurologic deficit” relates to tools designed to measure and scale the neurological deficits most often seen with stroke. Several aspects or parameters are assessed, such as the level of consciousness, visual fields, facial weakness, motor performance of extremities, gaze, sensory deficits, coordination (ataxia), language (aphasia), speech (dysarthria), etc. For all of them a value is given, being 0 if normal. So, in most of these tools the higher the score, the worse the neurological deficit. The skilled man will know of the existence of different tools for this purpose.

Thus, in yet a more particular embodiment of the methods of any of the first or second aspects of the invention, the score from systematic assessment tools of stroke-related neurologic deficits is selected from National Institutes of Health Stroke Scale (NIHSS) score, the Rapid Arteria occlusion evaluation scale for stroke (RACE), the Cincinnati Prehospital Stroke Scale Compared to Stroke Severity Tools for Large Vessel Occlusion Stroke Prediction (Cincinnati-score or CPSS), Los Angeles Motor Scale (LAMS), Vision-Aphasia-Neglect (VAN), Field Assessment Stroke Triage for Emergency Destination (FAST-ED), or the modified Rankin Scale or Score (mRS).

Publications disclosing these tools include, for example, the one of Noorian A R, Sanossian N, Shkirkova K, Liebeskind D S, Eckstein M, Stratton S J, Pratt F D, Conwit R, Chatfield F, Sharma L K, Restrepo L, Valdes-Sueiras M, Kim, Tenser M, Starkman S, Saver J L; FAST-MAG Trial Investigators and Coordinators. Los Angeles Motor Scale to Identify Large Vessel Occlusion: Prehospital Validation and Comparison With Other Screens. Stroke. 2018 March; 49(3):565-572. Another publication is that of Gropen T I, Boehme A, Martin-Schild S, Albright K, Samai A, Pishanidar S, Janjua N, Brandler E S, Levine S R. Derivation and Validation of the Emergency Medical Stroke Assessment and Comparison of Large Vessel Occlusion Scales. J Stroke Cerebrovasc Dis. 2018 March; 27(3):806-815.

The term “NIHSS score”, as used in the present invention refers to The National Institutes of Health Stroke Scale (NIHSS) score, a systematic assessment tool that provides a quantitative measure of stroke-related neurologic deficit. The NIHSS was originally designed as a research tool to measure baseline data on patients in acute stroke clinical trials. Now, the scale is also widely used as a clinical assessment tool to evaluate acuity of stroke patients, determine appropriate treatment, and predict patient outcome. The NIHSS is a 15-item neurologic examination stroke scale used to evaluate the effect of acute cerebral infarction on the levels of consciousness, language, neglect, visual-field loss, extraocular movement, motor strength, ataxia, dysarthria, and sensory loss. A trained observer rates the patient's ability to answer questions and perform activities. Ratings for each item are scored with 3 to 5 grades with 0 as normal, and there is an allowance for untestable items. The level of stroke severity as measured by the NIH stroke scale scoring system: 0=no stroke, 1-4=minor stroke, 5-15=moderate stroke, 15-20=moderate/severe stroke, 21-42=severe stroke. In the present invention the term “higher score” refers to a score from 5 to 42 in the NIH stroke scale scoring system.

Also, variations of the NIHSS such as Rapid Arteria occlusion evaluation scale for stroke (RACE) or other scores used to identify ischemic strokes, such as CPSS with large vessel occlusion may be used.

In another particular embodiment of the in vitro method for the diagnosis of large vessel occlusion (LVO), or the in vitro method for selecting a subject for a reperfusion therapy, the method comprises determining the level of one or more of the following proteins: a natriuretic peptide, including one or more of B-type natriuretic peptide (BNP) and atrial natriuretic peptide (ANP); DDi, and GFAP.

In a particular embodiment, optionally in combination with any of the embodiments of the aspects above or below, the natriuretic peptide is a B-type natriuretic peptide. In even a more particular embodiment the BNP determined in the isolated sample of a subject is the prohormone called The N-terminal fragment of B-type natriuretic peptide (NT-proBNP), which is a secreted biologically inactive form of BNP. The N-terminal fragment of B-type natriuretic peptide is the 76-amino acid N-terminal fragment of the B-type natriuretic peptide prohormone. Cleaving of pro-BNP yields the NT-proBNP fragment and the active B-type natriuretic peptide (BNP). BNP is a hormone secreted by cardiomyocytes in the heart ventricles in response to stretching caused by increased ventricular blood volume. The complete human sequence BNPhas the UniProt KB accession number P16860 (Aug. 1, 1990—version 1 of the sequence, and database release 187 of May 8, 2019). To simplify, in this description NT-proBNP and BNP are used as synonymous abbreviations, although in the examples below NT-proBNP was the form finally determined in the isolated samples.

The term “DDi” relates to D-dimer (or D dimer), which is a fibrin degradation product (or FDP), a small protein fragment present in the blood after a blood clot is degraded by fibrinolysis. It is so named because it contains two D fragments of the fibrin protein joined by a cross-link. D-dimer concentration may be determined by a blood test to help diagnose thrombosis.

The term “GFAP” as used herein refers to glial fibrillary acidic protein, an intermediate filament protein that is expressed by numerous cell types of the central nervous system. The complete human sequence for glial fibrillary acidic protein has the UniProtKB accession number P14136 (Jan. 1, 1990-version 1 of the sequence, and database release 221 of Apr. 7, 2021)).

In a more particular embodiment of the methods, they comprise determining the level of one or two of the previously listed proteins in combination with the levels of a FABP, which is in particular HFABP. In a more particular embodiments of these methods including the determination of the levels of a FABP, in particular HFABP, and of one or two of the other proteins listed above, the method comprises also determining one or two or more clinical parameters of the subject.

Inventors realized that a value of 100% specificity for correct classification of an LVO from non-LVO subjects could be fixed with certain cut-offs/reference when determining the levels of HFABP in the isolated sample in combination with one or more clinical parameters of the previous list (see examples below). This fixed specificity gave sensitivities for detecting positive LVO from the population (true positives) around 50%. If in addition, the levels of one or two of the previously listed proteins are considered (determined), 100% of specificity with around 75% of sensitivity is obtained.

In another particular embodiment of the methods of the first and the second aspects of the invention, optionally in combination with any of the particular embodiments above or below, the said methods comprise determining at least one of the following combinations of markers (i.e., levels of HFABP and other proteins in the isolated sample of the subject and/or considering in the decision one or more clinical parameters of the subject):
(a) HFABP+NIHSS+DBP+age;
(b) HFABP+NT-proBNP+NIHSS+DBP+age;
(c) HFABP+DDi+NIHSS+DBP+age;
(d) HFABP+GFAP+NIHSS+DBP+age
(e) HFABP+NT-proBNP+DDi+NIHSS+DBP+age;
(f) HFABP+NT-proBNP+GFAP+NIHSS+DBP+age;
(g) HFABP+GFAP+DDi+NIHSS+DBP+age;
(h) HFABP+NIHSS+SBP+age;
(i) HFABP+NIHSS+mean BP+age; and
(j) HFABP+NTproBNP+NIHSS+mean BP+age.

As will be illustrated in the examples, most of these combinations including determining the levels of HFABP, and in particular combinations (a)-(g), provided sensitivities for LVO diagnosis higher than 40%, even higher than 70%, in combination with specificities higher than 90%, even of 100%.

The combination of the levels of HFABP with the scores of also another systematic assessment tool of stroke-related neurologic deficits (Cincinnati-CPSS), provides also high sensitivities for LVO diagnosis in combination with high specificities. Therefore, in another particular embodiment of the methods of the first and the second aspects of the invention, optionally in combination with any of the particular embodiments above or below, the said methods comprise determining at least one of the following combinations of markers (i.e., levels of HFABP and other proteins in the isolated sample of the subject and/or considering in the decision one or more clinical parameters of the subject):
(k) HFABP+Cincinnati+DBP+age;
(l) HFABP+Cincinnati+SBP+age;
(m) HFABP+Cincinnati+mean BP+age;
(n) HFABP+NT-proBNP+Cincinnati+DBP+age;
(o) HFABP+DDi+Cincinnati+DBP+age;
(p) HFABP+GFAP+Cincinnati+DBP+age;
(q) HFABP+NT-proBNP+DDi+Cincinnati+DBP+age;
(r) HFABP+NT-proBNP+GFAP+Cincinnati+DBP+age;
(s) HFABP+GFAP+DDi+Cincinnati+DBP+age; and
(t) HFABP+NTproBNP+Cincinnati+mean BP+age

In yet another particular embodiment, when the in vitro methods of the invention comprise determining the level of one or more of DDi, GFAP, a natriuretic peptide, such as NT-proBNP, in the isolated sample of the subject, and/or determining the one or more clinical parameters as above disclosed, the subject is diagnosed of suffering from LVO:
when the level of a FABP, in particular HFABP, is equal or within a range of a reference of a subject suffering from LVO and the level of one or more of the other listed proteins are also within a range of a reference of a subject suffering from LVO; and/or
when the level of a FABP, in particular HFABP is equal or within a range of a reference of a subject suffering from LVO and the values of the one or more of the clinical parameters is also within a range of a reference of a subject suffering from LVO; and/or
wherein the method further comprises the step of computing all the values of the determined levels of the proteins and of the determined clinical parameters in a formula to obtain a predictive factor, and diagnosing the subject is suffering from LVO when this predictive factor is within the values of a reference (i.e., cut-off) or interval of LVO.

These methods including the determining of one or more levels of proteins in the isolated sample and/or of considering in the decision one or more clinical parameters of the subject in combination with the levels of FABP, such as HFABP, provide more accurate classification of the patient, including higher sensitivities and specificities.

The term “reference value”, as used herein, relates to a predetermined criterion used as a reference for evaluating the values or data obtained from the samples collected from a subject. The reference value or reference level can be an absolute value (i.e., a cut-off value or cut-off discriminating value); a relative value; a value that has an upper or a lower limit; a range of values (i.e., a range of possible cut-off values); an average value; a median value, a mean value, or a value as compared to a particular control or baseline value. A reference value or reference range can be based on an individual sample value, such as for example, a value obtained from a sample from the subject being tested, but at an earlier point in time. The reference value or range can be based on a large number of samples, such as from population of subjects of the chronological age matched group, or based on a pool of samples including or excluding the sample to be tested. Reference values have been determined for the biomarkers of the invention. The reference value for a FABP, such as HFABP, may be from a lower and an upper value as will be disclosed in view of examples below. Range of values of each biomarker (protein levels) and particular combinations of the values of the different biomarkers provide for correct classification of subjects with high sensitivity and specificity.

Indeed, the different alternative embodiments of the methods of the aspects disclosed according to the invention when including the option of comparing tested levels with respective cut-off values or reference intervals, these values or intervals may vary, and they are selected considering particular values of desired sensitivities and specificities. Thus, if a 100% specificity (true negative or correct classification between two conditions) is desired, sensitivity (true positives or detection of one condition among a cohort of subjects with different conditions) can be lowered. On the other hand, lowering the specificity (i.e., around 94% or 98%) allows generally increasing sensitivity of a method. Therefore, reference values/cut-offs can be varied depending on the desired specificity and/or sensitivity desired. For example, for a particular pair of specificity and sensitivity a cut-off is appropriate, but for a different pair of specificity and sensitivity a different cut-off is considered as reference. In the same way, particular cut-offs may indicate or discriminate a condition among several different ones, and other cut-offs may give additional information about the same condition, for example a prognostic information.

In the present case, cut-offs for the HFABP, as an example of FABP family member, selected from the interval defined from 1.25 ng/ml to 6.0 ng/ml in blood have been determined informative for the correct diagnosis of LVO and even for different features of this LVO condition. In particular cut-offs of HFABP levels in blood of 1.25, 1.50, 1.75, 2.00, 2.25, 2.50, 2.75, 3.0, 3.25, 3.50, 3.75, 4.0, 4.25, 4.50, 4.75, 5.0, 5.25, 5.50, 5.75, 6.0 ng/ml.

Furthermore, the reference values or intervals may vary in function of the characteristics of the subject, such as the subject race or even the ethnic/geographical genetic background of the subject. Reference values (i.e., cut-offs) in the examples below have been determined for Caucasian race subjects, in particular from cohorts of people in Spain.

In summary, the absolute value of a reference value or absolute group of values in an interval will be adjusted according to certain parameters, but the relevance of the determining of a FABP, such as HFABP, alone or in combination with other parameters or levels of other proteins is that a decision can be taken with confidence by the comparison of the levels of FABP (i.e., HFABP) with said references.

Indeed, the accuracy of statistical methods used in accordance with the present invention can be best described by their receiver operating characteristics (ROC). The ROC curve addresses both the sensitivity, the number of true positives, and the specificity, the number of true negatives, of the test. Therefore, sensitivity and specificity values for a given combination of biomarkers are an indication of the accuracy of the assay. For example, if a biomarker combination has sensitivity and specificity values of 80% for LVO, out of 100 patients which have and LVO ischemic stroke, 80 will be correctly identified from the determination of the presence of the particular combination of biomarkers as positive for LVO ischemic stroke (LVO IS), while out of 100 patients who have not suffered an LVO ischemic stroke (non-LVO IS) 80 will accurately test negative for the disease. In other words, sensitivity of the method provides information about the true positive subjects or proportion of subjects for LVO correctly detected from a group. Specificity of the method provides information about the true negative subjects or proportion of subjects correctly rejected from the group. Thus, in the context of the invention, a specificity of 100% provided by a method means that all non-LVO subjects will be rejected, which means that only most (80%) of the actual LVO patients will be correctly selected for the particular and specific adequate treatment. Thus, time and efforts will not be lost trying to treat a non-LVO with a non-adequate treatment (non-LVO do not need a thrombectomy, for example).

The levels of a bio marker (in this invention the level of HFABP as a FABP family member or of any other of the listed proteins) are considered to be higher than its reference value or range when it is at least 1.5%, at least 2%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%: at least 85%, at least 90%, at least 95%, at least 100%, at least 110%, at least 120%, at least 130%, at least 140%, at least 150% or more higher than the reference value.

Likewise, in the context of the present invention, the level of a biomarker is reduced when the level of said biomarker in a sample is lower than a reference value. The levels of a biomarker are considered to be lower than its reference value when it is at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%: at least 85%, at least 90%, at least 95%, at least 100%, at least 110%, at least 120%, at least 130%, at least 140%, at least 150% or more lower than the reference value.

The term “predictive factor”, as used herein, refers to a factor that is derived from the determined levels of the above-mentioned proteins, optionally in combination with one or more clinical parameters. The predictive factor can be calculated by summing the values obtained for each protein, and/or the values of the clinical parameters, corrected by a particular coefficient for each of the proteins and/or for the clinical parameters. Statistical methods for calculating such correction coefficients are known to those skilled in the art. This “predictive factor” can also be obtained applying different algorithms known by the skilled person in the art. Examples of possible algorithms, including statistical/mathematical algorithms, are listed below in more detail.

Inventors have also realized that when the levels of HFABP (as an example of FABP) in the isolated sample indicate the subject is suffering and LVO, and said levels are at least four units increased in relation to the cut-off or reference for diagnosing LVO, the subject is effectively suffering LVO and can also be classified as a subject with a poor response to therapy in terms of the dependency degree after the stroke and the applied or recommended cure. In other words, these subjects with these very high levels of HFABP give a response to the applied therapy corresponding to an increase in the odds ratio of dependency at three months by 2.006 (p-value=0.040), hence duplicating the risk of a modified ranking score (an mRS) higher than 2.

Thus, in a more particular embodiment of the first aspect, when the subject is diagnosed of LVO it is also classified as a subject with a dependency degree greater than 2 according to modified ranking score (mRS), and determined within 1-5 months, after stroke onset (episode), preferably determined at 3 months after stroke, if the levels of HFABP in the isolated sample of the subject diagnosed of LVO are at least four units increased in relation to the cut-off or reference for diagnosing LVO.

This particular embodiment of the diagnosis method also applies to the method for selecting a subject for thrombectomy (i.e., the most likely resolutive reperfusion in these patients). Note that thrombectomy will be recommended even in case of a likely bad or poor response to therapy, mainly for ethical code.

Indeed, linked with this observation regarding the type of response to therapy of the subjects suffering from an ischemic stroke with an LVO, inventors are disclosing a method for the prediction of response of a patient suffering ischemic stroke with an LVO, and thus candidates to reperfusion therapy, in particular thrombectomy, which method comprises determining the level of expression of HFABP (as an example of a FABP), in an isolated sample of the patient, and then comparing the levels with a reference value (i.e., cut-off) allowing to classify the subject as suffering LVO, being levels at least four units increased in relation to the said cut-off or reference for diagnosing LVO indicative of a type of response leading or evolving to a dependency degree greater than 2 according to modified ranking score (mRS), and determined within 1-5 months after stroke onset. According to the best of inventor's knowledge, this is the first time this bad response to therapy association with HFABP for an LVO has been indicated.

The inventors have also realized that HFABP is a marker that allows the implementation of a fast decision for the intervention in hospitals at a specialized and instrumented rooms for thrombectomy; the so-called angio-suites or angiographic rooms or angio-suites.

Angio-suite are areas at hospitals were catheter intervention and stenting are performed. These rooms include diagnostic imaging equipment used to visualize the arteries of the heart or the brain and then to treat any stenosis or abnormality (i.e. a LVO) found.

Thus, as previously formulated, another aspect of the invention is an in vitro method for selecting a subject to be shifted to a centre for thrombectomy and/or for a direct transfer to an angio-suite in the said centre for thrombectomy, the method comprising determining the level of a fatty acid binding protein (FABP) in an isolated sample of the subject, and comparing the said level with a reference value or range, wherein:
(a) the subject is selected to be shifted to a centre for thrombectomy when the level of FABP in the isolated sample is equal to a reference value or within a range of a subject suffering from LVO; or
(b) the subject is selected to be shifted to a centre for thrombectomy when the level of FABP in the isolated sample is higher than a reference value or range of a subject not suffering from LVO; and/or
(c) the subject is selected for a direct transfer to an angio-suite in the said centre for thrombectomy, when the level of FABP in the isolated sample is higher than a reference value that allows discriminating with a specificity of 100% between a subject suffering from either an LVO or a hemorrhagic stroke from a subject suffering from either a non-LVO ischemic or a mimic stroke.

In a particular embodiment of the method for selecting a subject to be shifted to a centre for thrombectomy and/or for the direct transfer to an angio-suite in the said centre for thrombectomy, FABP is selected from HFABP, BFABP, and combinations thereof.

It is thus disclosed, in particular, a method for the diagnosis of LVO and for the selection of a subject for thrombectomy with a previous (i.e., plane CT) scanning of the brain in a angio-suite, wherein the method comprises the step of determining the level of HFABP in an isolated sample of the subject, and comparing said level with a reference value (i.e., a cut-off values) that allows discriminating with a specificity of 100% between a subject suffering either an LVO or an haemorrhagic stroke from a subject suffering either a non-LVO IS or a mimic subject, and wherein if the levels are higher than this cut-off the subject is selected for the thrombectomy with a previous scanning of the brain in the angio-suite.

The rationale behind this method is that, although a subject with a haemorrhagic stroke will not be selected for a thrombectomy, the haemorrhage can be detected by means of the brain scanning instruments in the angio-suite, which is equipped with at least a plane computerized tomography (CT) scan. If after this scan at angio-suite no haemorrhage is detected, the subject is an LVO and he/she is already in the adequate room for the performance of the thrombectomy. This is finally translated to a faster treatment of even from 2 hours to 2:30 hours faster than whether the subject would had first gone to the emergency department of the hospital for having first a more complex but accurate brain scanning by cerebral angiography. If the scan at angio-suite reveals the existence of haemorrhage, the subject is then derived to a corresponding neurosurgery or stroke unit area in the hospital.

Thus, this method for selecting a subject for a thrombectomy centre and/or for a direct shift to an angio-suite, is indeed within the methods of any of the first and second aspects with the particularity that the selected reference cut-off allows the classification to speed the treatment of the actual LVO ischemic stroke.

In another particular embodiment of any of the methods of previous aspects, optionally in combination with any embodiment above or below, when the subject is diagnosed of LVO, the subject is also classified as:
(i) having a prognosis defined by a dependency degree greater than 2 according to modified ranking score (mRS), and determined within 1-5 months after stroke onset; and/or
(ii) having a prognosis defined by a three-month after onset mortality rate comprised from 25%-35%,
if the level of FABP is higher than a cut-off value stratifying the patients according to either the dependency degree and/or the mortality rate.

This prognostic value associated with the diagnosis of LVO is a noteworthy advantage and it is exemplified with a representative cohort in the examples.

The term “sample” as used herein, relates to any sample which can be obtained from the patient (subject). The present method can be applied to any type of biological sample from a patient, such as a biopsy sample, tissue, cell or biofluid (plasma, serum, saliva, semen, sputum, cerebral spinal fluid (CSF), tears, mucus, sweat, milk, brain extracts and the like.

In another particular embodiment of any of the methods of previous aspects, optionally in combination with any embodiment above or below, the isolated sample of the subject (i.e. patient suffering stroke) is a bio fluid. Illustrative non limitative bio fluids are blood (i.e., whole blood), plasma, serum, saliva, urine or cerebrospinal fluid. In a more preferred embodiment, the biofluid is plasma or serum. In another more preferred embodiment, the biofluid is whole blood obtained either by fingerprinting or by collection in a tube.

Different samples could be used for determining the level of different markers. Thus, it is not necessary that the levels of all the markers according to the methods of the invention are measured in the same type of sample. Thus, in another particular embodiment, the levels of HFABP (as an example of FABP) and if determined of the other proteins listed (i.e., GFAP, DDi, BNP . . . ) are measured in serum or in whole blood. In another particular embodiment, the level of any of them are measured in plasma.

In a preferred embodiment of the methods of the invention, the sample is obtained at baseline.

“Baseline”, as used in the present invention, is considered any time from onset of symptoms until the patient is explored for the first time. This is usually within the first hours after stroke, and it is usually the first attention in the ambulance or in the hospital. In a preferred embodiment, the baseline is within the first 4.5 hours from symptom onset, or less than 6 hours after stroke or in another preferred embodiment less than 24 hours from symptoms onset. In another particular embodiment, the levels of the markers, in particular of HFABP (as an example of FABP) and one or more of NT-proBNP (as example of natriuretic peptide), DDi, and GFAP, are determined within the first 24 hours from onset of symptoms, within the first 12 hours, within the first 6 hours or within the first 3 hours from stroke onset or symptoms. Inventors have realized that accurate classifications can be obtained considering the time of measure of the levels of HFABP in the isolated sample.

In another particular embodiment of any of the first and second aspects of the invention, or of any other aspects previously indicated, the in vitro methods comprise the determining of the levels of FABP, in particular of HFABP, and if determined, of the one or more of BNP (e.g., NT-proBNP), DDi, and GFAP within the first two hours after a stroke onset, more in particular within the first (1) hour after a stroke onset.

Thus, after a diagnosis of LVO is accomplished, in another particular embodiment of the first and second aspects it further comprises a step of recommending a reperfusion therapy, in particular a thrombectomy to the said diagnosed subject and/or treating said subject with a reperfusion therapy, in particular with a thrombectomy.

This particular embodiment could be drafted as a method of treating a subject suffering LVO, said method comprising carrying out the in vitro method for the diagnosis of LVO, in a patient according to the first aspect and treating a patient diagnosed of LVO with a reperfusion therapy, in particular with a thrombectomy. Advantageously, with this method patient is treated or recommended to be treated within first hours of the onset of symptoms and with the most appropriate therapy regimen.

Also encompassed herewith is, as another particular embodiment of the methods of the first and second aspects of the invention, optionally in combination with any embodiment above or below of these aspects, that it further includes a step of treating the subject with said reperfusion therapy, such as a thrombectomy if at least the level of a FABP, such as HFABP, is equal or within a range of a reference of a subject suffering from LVO or higher than a reference value of a subject not suffering from LVO.

This description also encompasses a method of detecting, in an isolated sample of a subject suffering from stroke coursing with LVO, the level of a FABP, such as HFABP, the method comprising:
(a) obtaining a sample from the subject; and
(b) detecting whether FABP, such as HFABP, is present in the isolated sample by: (i) contacting said sample with means capable of binding the corresponding expressed FABP (e.g., HFABP) and detecting said binding; or (ii) contacting said sample with means capable of binding corresponding RNA going to be translated to the said FABP (e.g., HFABP) and detecting said binding.

This method of detecting is, in a particular embodiment, carried out by also detecting in step (b) whether one or more of a BNP, such as NT-proBNP, DDi and GFAP is present in the isolated sample by: (i) contacting said sample with means capable of binding the corresponding expressed proteins and detecting said binding; or (ii) contacting said sample with means capable of binding corresponding RNA going to be translated to the one or more of the corresponding proteins and detecting said binding.

The determination of the levels of the proteins (i.e., HFABP and other proteins listed) in the isolated sample in all the in vitro methods of the invention can be carried out by qualitative and/or quantitative tests selected from the group consisting of an immunological test, bioluminescence, fluorescence, chemiluminescence, electrochemistry and mass spectrometry. Particular tests that can be implemented in a point of care test format (POCT) are recommended to make easy and fast the determining of marker levels. In a particular embodiment, point of care tests include lateral flow tests, which allow detecting the presence (or absence) of a target analyte in liquid sample (matrix) without the need for specialized and costly equipment, though many lab-based applications exist that are supported by reading equipment.

Independently of the test format, particular quantitative tests are selected from the group consisting of an immunological test, bioluminescence, fluorescence, chemiluminescence, electrochemistry and mass spectrometry.

In one embodiment, the level of expression is determined by immunological techniques such as enzyme-linked immunosorbent assay (ELISA), enzyme immunodot assay, agglutination assay, antibody-antigen-antibody sandwich assay, antigen-antibody-antigen sandwich assay, immunochromatography, or other immunoassay formats well-known to the ordinarily skilled artisan, such as radioimmunoassay, as well as protein microarray formats, such as single molecular assay (SIMOA), Western Blot or immunofluorescence.

Western blot is based on the detection of proteins previously resolved by gel electrophoreses under denaturing conditions and immobilized on a membrane, generally nitrocellulose by the incubation with an antibody specific and a developing system (e.g. chemiluminescent). The analysis by immunofluorescence requires the use of an antibody specific for the target protein for the analysis of the expression. ELISA is based on the use of antigens or antibodies labelled with enzymes so that the conjugates formed between the target antigen and the labelled antibody results in the formation of enzymatically-active complexes. Since one of the components (the antigen or the labelled antibody) are immobilised on a support, the antibody-antigen complexes are immobilised on the support and thus, it can be detected by the addition of a substrate which is converted by the enzyme to a product which is detectable by, e.g. spectrophotometry, fluorometry, mass spectrometry or tandem mass tags (TMT). SIMOA is a type of assay more sensitive than an ELISA, since it uses arrays of femtoliter-sized reaction chambers, which are termed single-molecule arrays (Simoa™) that can isolate and detect single enzyme molecules. Because the array volumes are approximately 2 billion times smaller than a conventional ELISA, a rapid build-up of fluorescent product is generated if a labelled protein is present. With diffusion defeated, this high local concentration of product can be readily observed. Only a single molecule is needed to reach the detection limit. Using the same reagents as a conventional ELISA, this method has been used to measure proteins in a variety of different matrices (serum, plasma, cerebrospinal fluid, urine, cell extracts, etc.) at femtomolar (fg/mL) concentrations, offering a roughly 1000-fold improvement in sensitivity.

On the other hand, the determination of the protein levels can be carried out by constructing a tissue microarray (TMA) containing the subject samples assembled, and determining the expression levels of the proteins by techniques well known in the state of the art.

In a preferred embodiment the determination of the levels of the markers are determined by immunological technique. In a more preferred embodiment, the immunological technique is ELISA.

When an immunological method is used, any antibody or reagent known to bind with high affinity to the target proteins (i.e., HFABP and optionally of the others listed above) can be used for detecting the amount of target proteins. It is preferred nevertheless the use of antibody, for example polyclonal sera, hybridoma supernatants or monoclonal antibodies, antibody fragments, Fv, Fab, Fab′y F(ab′)2, ScFv, diabodies, triabodies, tetrabodies and humanised antibodies.

As the person skilled in the art understands, the expression levels of a FABP such as HFABP and, if determined, of the other proteins listed, can be detected by measuring the levels of mRNA encoded by the corresponding genes.

By way of a non-limiting illustration, the expression levels are determined by means of the quantification of the levels of mRNA encoded by said genes. The latter can be quantified by means of using conventional methods, for example, methods comprising the amplification of mRNA and the quantification of the amplification product of said mRNA, such as electrophoresis and staining, or alternatively, by means of Northern blot and the use of suitable probes, Northern blot and use of specific probes of the mRNA of the genes of interest or of their corresponding cDNA/cRNA, mapping with the SI nuclease, RT-PCR, hybridization, microarrays, etc. Similarly, the levels of the cDNA/cRNA corresponding to said mRNA encoded by the marker genes can also be quantified by means of using conventional techniques; in this event, the method of the invention includes a step of synthesis of the corresponding cDNA by means of reverse transcription (RT) of the corresponding mRNA followed by the synthesis (RNA polymerase) and amplification of the cRNA complementary to said cDNA. Conventional methods of quantifying the expression levels can be found in laboratory manuals.

In order to normalize the values of mRNA expression among the different samples, it is possible to compare the expression levels of the mRNA of interest in the test samples with the expression of a control RNA. A “control RNA” as used herein, relates to RNA whose expression levels do not change or change only in limited amounts. Preferably, the control RNA is mRNA derived from housekeeping genes and which code for proteins which are constitutively expressed and carry out essential cellular functions. Preferred housekeeping genes for use in the present invention include 18-S ribosomal protein, ß-2-microglobulin, ubiquitin, cyclophilin, GAPDH, PSMB4, tubulin and B-actin.

In another particular embodiments of the in vitro methods of the invention that provide a diagnostic of LVO and information for selecting a therapy, they further comprise the steps of (i) collecting the diagnostic information, and (ii) saving the information in a data carrier.

In the sense of the invention a “data carrier” is to be understood as any means that contain meaningful information data for the diagnosis of LVO and/or for the selection of a candidate to reperfusion therapy (e.g., a thrombectomy), such as paper. The carrier may also be any entity or device capable of carrying the diagnosis data or information for selecting a therapy. For example, the carrier may comprise a storage medium, such as a ROM, for example a CD ROM or a semiconductor ROM, or a magnetic recording medium, for example a floppy disc or hard disk. Further, the carrier may be a transmissible carrier such as an electrical or optical signal, which may be conveyed via electrical or optical cable or by radio or other means. When the diagnosis/therapy selection data are embodied in a signal that may be conveyed directly by a cable or other device or means, the carrier may be constituted by such cable or other device or means. Other carriers relate to USB devices and computer archives. Examples of suitable data carrier are paper, CDs, USB, computer archives in PCs, or sound registration with the same information.

As previously indicated, the invention also relates to kits comprising reagent means for detecting the level of one or more member of the FABP family.

More in particular the kits comprise reagent means for detecting simultaneously the level of one or more member of the FABP family, and of one or more of the levels of a natriuretic peptide, such as ANP or BNP (in particular NT-proBNP), DDi, and GFAP.

In a particular embodiment of the kit, it comprises reagent means for detecting simultaneously the level of HFABP and of the level of one or more of the levels of NT-proBNP, DDi, and GFAP.

Additionally, the kits of the invention can contain instructions for the simultaneous, sequential or separate use of the different components which are in the kit. Said instructions can be in the form of printed material or in the form of an electronic support capable of storing instructions susceptible of being read or understood, such as, for example, electronic storage media (e.g., magnetic disks, tapes), or optical media (e.g., CD-ROM, DVD), or audio materials. Additionally, or alternatively, the media can contain internet addresses that provide said instructions.

The reagent means (or simply reagents) of the kit include compounds that specifically bind to the protein of interest which levels in the isolated sample are to be determined. In another more particular embodiment of the kits of the invention, said compounds that specifically bind to the protein are selected from an antibody, an aptamer, a fragment of any of the antibody or of the aptamer and combinations thereof.

These antibodies or aptamers, or their fragments, specifically recognize a FABP, such as HFABP. The other antibodies of the kit will specifically recognize the other proteins, if determined in the corresponding particular embodiments of the methods of the first and second aspects. The antibodies of the kit of the invention can be used according to techniques known in art for determining the protein expression levels, such as, for example, flow cytometry, Western blot, ELISA, RIA, competitive EIA, DAS-ELISA, techniques based on the use of biochips, protein microarrays, or assays of colloidal precipitation in reactive strips.

The antibodies can be fixed to a solid support such as a membrane, a plastic or a glass, optionally treated to facilitate the fixation of said antibodies to the support. Said solid support comprises, at least, a set of antibodies which specifically recognize the marker (i.e., the protein of interest), and which can be used for detecting the levels of expression of said marker.

Additionally, the kits of the invention comprise reagents for detecting a protein encoded by a constitutive gene. The availability of said additional reagents allows normalizing the measurements performed in different samples (for example, the sample to be analysed and the control sample) to rule out that the differences in the expression of the biomarkers are due to a different quantity of total protein amount in the sample more than the real differences in the relative levels of expression. The constitutive genes in the present invention are genes that are always active or being transcribed constantly and which encode for proteins that are expressed constitutively and carry out essential cellular functions. Proteins that are expressed constitutively and can be used in the present invention include, without limitation, β-2-microglobulin (B2M), ubiquitin, 18-S ribosomal protein, cyclophilin, GAPDH, PSMB4, tubulin and actin.

In a preferred embodiment, the reagent means for assaying the levels of the different biomarkers (proteins) comprise at least 10%, at least 20%, at least 30%, at least 40%, at least 50%, at least 60%, at least 70%, at least 80%, at least 90% or at least 100% of the total amount of reagents for assaying biomarkers forming the kit. Thus, in the particular case of kits comprising reagents for assaying the levels of HFABP (or of any other FABP member of the family) and of one or more of a natriuretic peptide (e.g. BNP such as NT-proBNP and/or ANP), DDi and GFAP, the reagents specific for said biomarkers (i.e. antibodies which bind specifically to the proteins) comprise at least 10%, at least 20%, at least 30%, at least 40%, at least 50%, at least 60%, at least 70%, at least 80%, at least 90% or at least 100% of the antibodies present in the kit. These kits are, thus, simplified kits including mainly the reagent means for detecting the levels of the HFABP (or of any other FABP member of the family) and of the one or more of a natriuretic peptide selected from an ANP and BNP such as NT-proBNP; DDi; and GFAP. In particular, reagent means for assaying the levels of HFABP and of one or two of a natriuretic peptide (e.g., NT-proBNP), DDi and GFAP.

In a particular embodiment of the kits, they comprise as single reagent means (i.e., antibodies, aptamers, fragments) for detecting the proteins, a compound that specifically binds HFABP (or any other FABP member of the family) and a compound that specifically binds DDi (or any detectable isoform of DDi). In another embodiment, the kit comprises as single reagent means for detecting the proteins, a compound that specifically binds HFABP (or any other FABP member of the family) and a compound that specifically binds GFAP (or any detectable isoform of GFAP). In another embodiment, the kit comprises as single reagent means for detecting the proteins, a compound that specifically binds HFABP (or any other FABP member of the family or isoforms) and a compound that specifically binds a natriuretic peptide, in particular selected from a BNP and an ANP. In another embodiment, the kit comprises as single reagent means for detecting the proteins, a compound that specifically binds HFABP (or any other detectable FABP member of the family), a compound that specifically binds DDi (or any detectable isoform), and a compound that specifically binds a natriuretic peptide, in particular selected from a BNP and an ANP. In another embodiment, the kit comprises as single reagent means for detecting the proteins, a compound that specifically binds HFABP (or any other detectable FABP member of the family or isoforms thereof), a compound that specifically binds DDi (or any detectable isoform), and a compound that specifically binds GFAP (or any detectable isoform). In another embodiment, the kit comprises as single reagent means for detecting the proteins, a compound that specifically binds HFABP (or any other detectable FABP member of the family or isoforms thereof), a compound that specifically binds GFAP, and a compound that specifically binds natriuretic peptide, in particular selected from a BNP and an ANP, or any isoforms thereof.

In the previous paragraph the isoforms of the listed proteins relate to any form of the expressed proteins of interest for which an assay is available and meanwhile this isoform is applicable to the method for the diagnosis or selection of therapy in the sense that gives the appropriate information.

In another particular embodiment, the kits of the invention are conceived as point of care tests. More in particular they are in form of lateral flow tests.

In another particular embodiment the kit according to the invention comprises a support and one or more sample inlet ports for deposition of a biofluid sample, in particular whole blood; a reaction area comprising the means/reagents that bind specifically to the marker proteins, in particular antibodies; and wherein the sample inlet port is connected with the reaction area. In another more particular embodiment, the kit comprises as many sample inlet ports as markers (one, two or three) to be detected and corresponding reaction areas connected thereto. In another embodiment the kit comprises one single inlet import and as capillary tracks connecting to as many reactive areas, said capillary tracks conducting part of the sample to each corresponding connected reaction area. The kits comprising more than one reaction areas are multiplex kits.

In another aspect, the invention relates to the use of the kit of the invention for diagnosing LVO or for selecting a patient suffering stroke for a thrombectomy or for any other adequate reperfusion therapy, such as a therapy with an antithrombotic agent.

Thus, in a particular embodiment, the invention relates to the use of the kit of the invention in any of the methods of the invention.

Inventors have also determined another combination of markers that allow an accurate diagnosis of LVO in a subject suffering from an ischemic stroke. It is in particular the combination including determining, in the isolated sample of a subject, the levels of a natriuretic peptide, in particular BNP (e.g., Nt-proBNP), GFAP and DDi. High sensitivities accompanied with high specificities were determined.

In a more particular embodiment of this working combination of markers, the levels of NT-proBNP, GFAP and DDi are determined in the isolated sample and then computed in combination with one or more clinical parameters. In particular they are computed in combination with the values of a NIHSS, the diastolic blood pressure (DBP) and the age of the subject. With particular cut-offs for any of the proteins, said cut-offs being higher than a reference value of a non-LVO subject (i.e., health subject) for each of the proteins, sensitivities around 80% were achieved with specificities around 95%. More in particular, when the cut-offs and values of NIHSS and of age and DBP were as follow:

Thresholds (cut-offs): NT-proBNP>1385.5, DDI>359.289, GFAP>52.1995, NIHSS>9.5, Age>84.5, DBP<90.5. SE=78.9%, SP=94.3%.

This combination also allows, thus, the selection of the subject to be selected for a reperfusion therapy, in particular for a thrombectomy. Thus, kits and means for carrying out the method for the diagnosis of LVO with this combination are also disclosed.

Inventors also determined that with the levels of HFABP in combination with certain clinical parameters an accurate classification between a subject suffering from ischemic stroke or suffering from any other condition selected from haemorrhagic stroke, transient ischemic attack or stroke mimicking conditions could be done. This accurate classification also allows a method for the selection of a candidate to a reperfusion therapy if the subject is classified as suffering from ischemic stroke.

Therefore, it is also disclosed an in vitro method for differentiating ischemic stroke from any other condition selected from haemorrhagic stroke, transient ischemic attack or stroke mimicking conditions in a subject, comprising determining the level of a FABP, in particular HFABP, in an isolated sample of the subject, in combination with one or more clinical parameters selected from the group consisting of blood pressure, including systolic blood pressure (BSP) and/or diastolic blood pressure (DBP), mean blood pressure (mean BP), glycemia, age, scores from systematic assessment tools of stroke-related neurologic deficits, time from onset of symptoms, and gender; the method further comprising the step of comparing the levels of said FABP, in particular of HFABP, and of the one or more of the clinical parameters with a corresponding reference value or interval of a subject suffering from ischemic stroke and/or from a subject suffering from any of haemorrhagic stroke, TIA or mimic stroke (i.e., a stroke mimicking condition); and wherein the subject is diagnosed as suffering from an ischemic stroke when the level of FABP, in particular of HFABP, and also the values of the one or more of the determined clinical parameters are within the value or interval of values from a subject suffering from ischemic stroke; or alternatively, the subject is diagnosed of any other condition selected from haemorrhagic stroke, transient ischemic attack or stroke mimicking conditions, when the level of FABP, in particular of HFABP, and also of the values of the one or more of the determined clinical parameters are within the value or interval of values from a subject suffering from any of the other conditions.

Another aspect of the invention is an in vitro method for differentiating ischemic stroke from any other condition selected from haemorrhagic stroke, transient ischemic attack or stroke mimicking conditions in a subject, comprising:
(a) determining the level of a FABP, in particular HFABP, in an isolated sample of the subject, in combination with:
   one or more clinical parameters selected from the group consisting of blood pressure, including systolic blood pressure (BSP) and/or diastolic blood pressure (DBP) and/or mean blood pressure (mean BP), glycemia, age, scores from systematic assessment tools of stroke-related neurologic deficits, time from onset of symptoms and gender; and
   the level of one or more of the following proteins: a natriuretic peptide, in particular selected from BNP and ANP; D-dimer (DDi); and GFAP in the isolated sample of the subject; and
(b) the step of comparing the levels of FABP, in particular of HFABP, and of the one or more of the clinical parameters and of the levels of the other proteins, with a corresponding reference value or interval of a subject suffering from ischemic stroke and/or from a subject suffering from any of an haemorrhagic stroke, TIA or mimic stroke; and wherein the subject is diagnosed as suffering from an ischemic stroke when the level of FABP, in particular of HFABP, and also of the one or more of the other determined proteins and/or the values of the one or more of the determined clinical parameters are within the value or interval of values from a subject suffering from ischemic stroke; or alternatively, the subject is diagnosed of suffering any other condition selected from an haemorrhagic stroke, a transient ischemic attack or a stroke mimicking condition, when the level of FABP, in particular of HFABP, and also of the one or more of the other determined proteins and/or the values of the one or more of the determined clinical parameters are within the value or interval of values from a subject suffering from any of the other conditions.

In a particular embodiment of the in vitro method for differentiating ischemic stroke from these other above-listed conditions, the method further comprises the step of computing all the values of the determined levels of the proteins and of the determined clinical parameters in a formula to obtain a predictive factor, and diagnosing the subject is suffering from ischemic stroke, and thus, as candidate to reperfusion, when this predictive factor is within the values of a reference or interval of ischemic stroke.

This step of computing all the values of the determined levels of the proteins and of the determined clinical parameters is also an alternative step (b) of the in vitro method for differentiating ischemic stroke from any of the above-listed other conditions (i.e. haemorrhagic stroke, TIA or mimic) according to the invention.

In another more particular embodiment of the in vitro method for differentiating ischemic stroke from the above-listed other conditions (i.e. hemorrhagic stroke, TIA or mimic) according to the invention, optionally in combination with any of the particular embodiments above or below, the method comprises determining at least one of the following combinations of markers (i.e., levels of HFABP and other proteins in the isolated sample of the subject and/or considering in the decision one or more clinical parameters of the subject):
(a) HFABP+NIHSS+DBP+age
(b) HFABP+NIHSS+mean BP+age
(c) HFABP+NT-proBNP+NIHSS+DBP+age
(d) HFABP+DDi+NIHSS+DBP+age
(e) HFABP+GFAP+NIHSS+DBP+age
(f) HFABP+NT-proBNP+DDi
(g) HFABP+NT-proBNP+DDi+NIHSS+mean BP+age
(h) HFABP+NT-proBNP+GFAP+NIHSS+mean BP+age
(i) HFABP+GFAP+DDi+NIHSS+mean BP+age
(j) HFABP+NT-proBNP+DDi+NIHSS+DBP+age;
(k) HFABP+NT-proBNP+GFAP+NIHSS+DBP+age;
(l) HFABP+GFAP+DDi+NIHSS+DBP+age
(m) HFABP+NIHSS+SBP+age;
(n) HFABP+Cincinnati+DBP+age;
(o) HFABP+Cincinnati+SBP+age;
(p) HFABP+Cincinnati+mean BP+age;
(q) HFABP+NT-proBNP+Cincinnati+DBP+age;
(r) HFABP+DDi+Cincinnati+DBP+age;
(s) HFABP+GFAP+Cincinnati+DBP+age;
(t) HFABP+NT-proBNP+DDi+Cincinnati+DBP+age;
(u) HFABP+NT-proBNP+GFAP+Cincinnati+DBP+age;
(v) HFABP+GFAP+DDi+Cincinnati+DBP+age;
(w) HFABP+NTproBNP+NIHSS+mean BP+age; and
(x) HFABP+NTproBNP+Cincinnati+mean BP+age.

As will be illustrated in the examples, all these combinations including determining the levels of HFABP, allowed providing sensitivities of 50-90% for the differential diagnosis, when 100% of specificity was fixed. These are advantageous features of the proposed method for the differential diagnosis, considering the gravity of a false positive for ischemic stroke (i.e., a true patient of haemorrhagic stroke) is finally treated with a reperfusion therapy.

All embodiments disclosed for the previous aspects regarding the in vitro method for the diagnosis of LVO, or the in vitro method for selecting a subject for a thrombectomy, also apply to this aspect of the differential diagnosis of ischemic stroke from haemorrhagic stroke.

Derived from this aspect, also disclosed is an in vitro method for the selection of a subject for a reperfusion therapy and/or for a therapy with neuroprotective drugs, the method comprising:
(a) determining the level of a FABP, in particular HFABP, in an isolated sample of the subject, in combination with:
   one or more clinical parameters selected from the group consisting of blood pressure, including systolic blood pressure (BSP) and/or diastolic blood pressure (DBP) and/or mean blood pressure (mean BP), glycemia, age, scores from systematic assessment tools of stroke-related neurologic deficits, time from onset of symptoms, and gender; and
   the level of one or more of the following proteins: a natriuretic peptide, in particular selected from BNP and ANP; D-dimer (DDi); and GFAP in the isolated sample of the subject; and
(b) the step of comparing the levels of FABP, in particular of HFABP, and of the one or more of the clinical parameters and of the levels of the other proteins, with a corresponding reference value or interval of a subject suffering from ischemic stroke and/or from a subject suffering from an haemorrhagic stroke, a transient ischemic attack or a stroke mimicking condition; and wherein the subject is diagnosed as suffering from an ischemic stroke and as candidate to a reperfusion therapy and/or candidate for a therapy with neuroprotective drugs, when the level of FABP, in particular of HFABP, and also of the one or more of the other determined proteins and/or the values of the one or more of the determined clinical parameters are within the value or interval of values from a subject suffering from ischemic stroke.

The neuroprotective therapy, mainly carried out by means of neuroprotective drugs includes the recommendation of administering to the subject diagnosed of an actual ischemic stroke (including LVO-IS and non-LVO IS), even at ambulance level, one or more of NA-1 (nerinetide), Uric acid, Activated protein C or 3K3A-APC-activated protein C, alfa-1-antitrypsin, fingolimod, metmorfin, glyburide, glibenclamide, TLR-4 inhibitors, IL1 inhibitors (IL1-RA, anakinra), nitric oxide donor, glyceryl trinitrate (GTN), cilostazol, gingko-biloba extracts and derivates, antioxidants, edaravone and edaravone derivates, resveratrol, melatonin, NAD, anti-intercellular adhesion molecule-1 (ICAM-1) antibodies, Enlimomab, calcium-stabilizing agents, and anti-excitotoxic agents, Maxipost (BMS-204352), Nalmefene (Cervene), Fosphenytoin, Enoxaparin, Trafermin, Ancrod, Magnesium, UK-279,276, ONO-2506, Dipyridamole, Repinotan, Simvastatin, Lubeluzole, Buspirone, Nimodipine, Heparin, YM872, Aptiganel (CNS-1102, Cerestat), Diazepam, Clomethiazole, Natalizumab, Ebselen, Flunarizine, Pentoxifylline, Abciximab, Pethidine, Dextromethorphan, Granulocyte colony stimulating factor (G-CSF) and other growth factors, pituitary adenylate cyclase-activating polypeptides, MMP-9 inhibitors, minocycline, or combinations of those or those and other related neurprotectants. The neuroprotective therapy also includes the recommendation of administering one or more of the previously listed compounds in combination with other neuroprotectants strategies such as hypothermia, remote limb ischemic postconditioning (RIPC), collateral cerebral blood flow augmentation, blood pressure manipulation, etc., that may be performed at the ambulance when the diagnostic test is done.

There are several therapy protocols for the promotion of reperfusion. In a particular embodiment of the first aspect of the invention, the reperfusion therapy is selected from the group consisting of a therapy with an antithrombotic agent, thrombectomy and a combination thereof.

In a more particular embodiment, the antithrombotic agent is a thrombolytic agent. In yet a more particular embodiment, the thrombolytic agent is a plasminogen activator. More in particular, the plasminogen activator is tissue plasminogen activator.

The term “antithrombotic agent”, as used herein, refers to a drug that is able to reduce clot formation. Suitable antithrombotic agents for use in the present invention include, without limitation, thrombolytic agents, antiplatelet agents and anticoagulant compounds.

The term “thrombolytic agent” as used herein refers to a drug that is able to dissolve a clot. All thrombolytic agents are serine proteases and convert plasminogen to plasmin which breaks down the fibrinogen and fibrin and dissolves the clot. Currently available thrombolyic agents include reteplase (r-PA or Retavase), alteplase (t-PA or Activase), urokinase (Abbokinase), prourokinase, anisoylated purified streptokinase activator complex (APSAC), staphylokinase (Sak), tenecteplase (TNKase® by Genenthec or TNK-tPA), atenecteplase (TNKasa), anistreplase (Eminase), streptoquinase (Kabikinase, Streptase) or uroquinase (Abokinase). Tenecteplase (TNK-tPA) is used in a particular embodiment, since it can be administered as a fast single bolus and can be used at ambulance level. TNK is effective after 1-minute post-administration (post-injection). Providers for TNK are Boehringer Ingelheim (European Union) and Genentech Inc (USA).

The term anticoagulant compounds, as used herein, refers to compounds that prevent coagulation and include, without limitation, vitamin K antagonists (warfarin, acenocumarol, fenprocoumon and fenidione), heparin and heparin derivatives such as low molecular weight heparins, factor Xa inhibitors such as synthetic pentasaccharides, direct thrombin inhibitors (argatroban, lepirudin, bivalirudin and ximelagatran) and antiplatelet compounds that act by inhibition of platelet aggregation and, therefore, thrombus formation and include, without limitation, cyclooxygenase inhibitors (aspirin), adenosine diphosphate receptor inhibitors (clopidogrel and ticlopidine), phosphodiesterase inhibitors (cilostazol), glycoprotein IIB/IIIA inhibitors (Abciximab, Eptifibatide, Tirofiban and Defibrotide) and adenosine uptake inhibitors (dipiridamol). In a preferred embodiment, the antithrombotic agent is a thrombolytic agent. In a more preferred embodiment, the thrombolytic agent is a plaminogen activator. In a yet more preferred embodiment, the plasminogen activator is tPA (tissue plasminogen activator).

The term “tissue plasminogen activator (t-PA)” as used herein refers to a serine protease found on endothelial cells that catalyzes the conversion of plasminogen to plasmin. The complete protein sequence for human t-PA has the UniProt KB accession number P00750 (Jul. 11, 2012). tPA may be manufactured using recombinant biotechnology techniques, tPA created this way may be referred to as recombinant tissue plasminogen activator (rtPA). Recombinant tissue plasminogen activators (r-tPAs) include the thrombolytic agents alteplase, reteplase, and tenecteplase (TNKase®, also termed TNK-tPA). In human t-PA, the amino acids at position 296-299 are lysine, histidine, and two arginine. In TNK-tPA, these amino acids have been replaced by four alanines. This mutation is responsible for increased resistance to plasminogen activator inhibitor 1 (PAI-1).

Doses of t-PA should be given within the first 3 hours of the onset of symptoms or up to 4.5 hours from symptom onset. Recommended total dose: 0.9 mg/kg (maximum dose should not exceed 90 mg) infused over 60 minutes. Load with 0.09 mg/kg (10% of the 0.9 mg/kg dose) as an intravenous bolus over 1 minute, followed by 0.81 mg/kg (90% of the 0.9 mg/kg dose) as a continuous infusion over 60 minutes. Heparin should not be started for 24 hours or more after starting alteplase for stroke. Said t-PA is given intravenously and in some cases may be given directly into an artery and should be given right away after the first symptoms of stroke start. Said doses and administration routes apply to any of the embodiments of the first aspect. Also, in particular in embodiments including step of treating the patient.

Single dose of TNK-tPA should be given as soon as possible after determining that the subject suffering from stroke is a candidate to reperfusion therapy, and within the first 3 hours of the onset of symptoms or up to 4.5 hours from symptom onset, preferably within the first hour after stroke onset.

As indicated above, the use of TNK-tPA is particularly useful, since due to the particular formulation as fast single application bolus, it can be administered at any point of care, even at ambulance level, being effective about one minute post-administration.

Those patients suffering stroke not selected for a reperfusion therapy, are in a particular embodiment, selected for a therapy reducing blood pressure. In particular, said therapy is performed with an agent capable of reducing blood pressure.

“Blood pressure” is herein to be understood as to refer to the blood pressure at the site of central arteries, such as the aorta and carotid artery. Central blood pressure can suitably be measured non-invasively (as set out below) at the carotids or radialis by applanation tonometry. “Blood pressure” as used herein thus encompasses aortic blood pressure.

“Agent capable of reducing blood pressure”, as used in the present invention, relates to any drug which lower blood pressure by different means. Among the most widely agents are the thiazide diuretics [such as furosemide, nitroprusside, hydralazine]; the ACE inhibitors, the calcium channel blockers (such as nicardipine or nimodipine); the adrenergic receptor antagonist (such as alpha-adrenergic antagonist, urapidil), or combined alpha- and beta-blocker (labetalol and nitroglycerin); and the angiotensin II receptor antagonists (ARBs). Illustrative, non-limitative example of agents capable of lowering or reducing blood pressure are alpha-methyl dopa (Aldomet), 11,17alpha-dimethoxy-18β-[(3,4,5-trimethoxy-benzoyl)oxyl)]-3p,2a-yohimban-16β-carboxylic acid methyl ester (Reserpine) or 2-(2,6-dichlorophenylamino) 2-imidazoline hydrochloride (Clonidine hydrochloride), lergotrile or viz. 2-chloro-6-methylergoline-8ß-acetonitrile as disclosed in EP0005074. Reference values that will be used to decrease blood pressure in ischemic stroke, ischemic stroke treated with thrombolytics or haemorrhagic stroke, will be those recommended by clinical practice guidelines as these values could be updated. Nowadays, treatment modalities for blood pressure lowering are aimed to be reduced if systolic blood pressure to among 220-120 mm Hg was achieved in ischemic patients and if it achieved to among 180-100 mm Hg in hemorrhagic patients. In a preferred embodiment, the blood pressure may be reduced by intravenous administration of an agent capable of reducing blood pressure and co-administration of oral antihypertensive agent(s). Reference values that will be used to decrease blood pressure in ischemic stroke, ischemic stroke treated with thrombolytics or haemorrhagic stroke, will be those recommended by clinical practice guidelines as these values could be updated.

Any method suitable for measure arterial pressure can be used for determining if an agent is capable of reducing blood pressure, wherein a reduction in arterial pressure is detected after administration of the agent. Illustrative, non-limitative examples of methods for measurement arterial pressure are non-invasive techniques, such as by way of illustrative non-limitative example palpitation, auscultatory, oscillometric and continuous non-invasive arterial pressure (CNAP).

Thus, after differential diagnosis is accomplished, in another particular embodiment of this differential method for ischemic stroke and/or for reperfusion and/or for neuroprotective therapy, it further comprises a step of recommending a reperfusion therapy and/or a therapy with neuroprotective drugs to a patient diagnosed of ischemic stroke and/or the treating of said patient diagnosed of ischemic stroke with a reperfusion therapy, mainly with an antithrombotic agent or by means of thrombectomy. On the alternative, those patients diagnosed of any other conditions, such as of an ICH that should avoid a reperfusion therapy in order to avoid fatal outcomes are, in another particular embodiment, recommended for or treated with a therapy reducing or optimizing blood pressure.

This particular embodiment could be drafted as a method of treating a patient suffering stroke, said method comprising carrying out the in vitro method for differentiating IS from other conditions, such as ICH in a patient above disclosed and treating a patient diagnosed of IS with a reperfusion therapy, mainly with an antithrombotic agent or by means of thrombectomy; or treating a patients diagnosed of other condition, in particular hose diagnosed of ICH, with a therapy reducing or optimizing blood pressure. Advantageously, with this method patient is treated or recommended to be treated within first hours of the onset of symptoms and with the most appropriate therapy regimen.

As previously commented for the methods of the first and second aspects, in a particular embodiment of the of the in vitro method for differentiating ischemic stroke from any other condition selected from a haemorrhagic stroke, a transient ischemic attack or a stroke mimicking condition according to the invention, it further comprises the steps of (i) collecting the diagnostic information, and (ii) saving the information in a data carrier, being the data carrier as previously defined.

Also, another aspect of the invention is an in vitro method for the prognosis of a patient suffering LVO, comprising determining the level of FABP in an isolated sample of said patient, and comparing the said level with a cut-off value stratifying the patients according to either the dependency degree and/or the mortality rate, wherein if the level of FABP in the sample is higher than the cut-off value, the subject suffering LVO is also classified as:
(i) having a prognosis defined by a dependency degree greater than 2 according to modified ranking score (mRS), and determined within 1-5 months after stroke onset; and/or
(ii) having a prognosis defined by a three-month after onset mortality rate comprised from 25%-35%.

In a particular embodiment of the in vitro method for the prognosis of a patient suffering LVO according to the previous aspect, FABP is selected from HFABP, BFABP, and combinations thereof.

In also another particular embodiment of the in vitro method for the prognosis, it further comprising determining one or more clinical parameters an/or features of the subject, said parameters in particular selected from the group consisting of blood pressure, including systolic blood pressure (SBP) and/or diastolic blood pressure (DBP), mean blood pressure (mean BP), glycemia, age, scores from systematic assessment tools of stroke-related neurologic deficits, time from onset of symptoms, gender, and combinations thereof.

As indicated for other aspect of the invention in which diagnosis of LVO is carried out, in a particular embodiment of the prognosis method, the score from systematic assessment tools of stroke-related neurologic deficits is selected from National Institutes of Health Stroke Scale (NIHSS) score, the Rapid Arteria occlusion evaluation scale for stroke (RACE), the Cincinnati Prehospital Stroke Scale Compared to Stroke Severity Tools for Large Vessel Occlusion Stroke Prediction (Cincinnati-score or CPSS), Los Angeles Motor Scale (LAMS), Vision-Aphasia-Neglect (VAN), Field Assessment Stroke Triage for Emergency Destination (FAST-ED), or the modified Rankin Scale or Score (mRS).

In also another particular embodiment of the in vitro method for the prognosis of subject previously diagnosed of LVO with the determining of FABP, in particular of HFABP, the method further comprises determining the level of one or more of a natriuretic peptide, in particular NT-proBNP; d-dimer (DDi); and glial fibrillary acid protein (GFAP), in the isolated sample of the subject.

All the embodiments applying to the first and second aspects of the invention regarding the time of measure and the type of sample do also apply to this other aspect regarding an in vitro method for the prognosis of a patient suffering LVO.

Finally, it is herewith provided an algorithm for carrying out any of the methods of diagnosis, and/or selection of a patient for a therapy and/or of prognosis as defined in the above aspects. In the sense of the invention, the term “algorithm” is also synonymous of panel or decision diagrams, predictors and combinatory of data to correctly categorize an individual sample.

According to aspects and embodiments of the invention, diagnosis of LVO, and/or the selection for a reperfusion therapy, and/or for discriminating among stroke types and sub-types is performed using a mathematical algorithm that assesses a detectable level of a FABP such as HFABP, and if determined of the other proteins, in particular detected by the means previously disclosed (i.e. antibodies or fragments thereof), either in conjunction with or independent of other clinical parameters, to correctly categorize an individual sample as originating from a healthy patient (i.e. mimics), a patient with an ischemic stroke with LVO, a patient with an ischemic stroke without an LVO (non-LVO IS), a patient with an haemorrhagic stroke.

The classification algorithm may be as simple as determining whether or not the amount of a specific biomarker or subset of biomarkers measured are above or below a particular cut-off number (or absolute value). When multiple biomarkers are used, the classification algorithm may be a linear regression formula. Alternatively, the classification algorithm may be the product of any of a number of learning algorithms. In the case of complex classification algorithms, it may be necessary to perform the algorithm on the data, thereby determining the classification, using a computer, e.g., a programmable digital computer. For example, and as above exposed, the algorithm is for carrying out the step of computing all the values of the determined levels of the proteins and of the determined clinical parameters in a formula to obtain a predictive factor, and diagnosing the subject is suffering from LVO when this predictive factor is within the values of a reference (i.e., cut-off) or interval of LVO, and then selecting the subject for a reperfusion therapy, in particular for a thrombectomy; or diagnosing the subject is suffering from ischemic stroke and not from an haemorrhagic stroke, TIA or mimics, if the predictive factor is within the values of a reference (i.e., cut-off) or interval of an ischemic stroke. In either case, one can then record the status on tangible medium, for example, in computer-readable format such as a memory drive or disk or simply printed on paper. The result also could be reported on a computer screen. This algorithm is used as diagnostic and/or prognostic method, and it is in particular part of the kits for carrying out the methods disclosed in former aspects.

The skilled person will be aware of numerous suitable methods for developing and applying statistical algorithms, and all of these are within the scope of the present invention. Examples of suitable classification algorithms include, as indicated, a linear regression, such as a logistic regression after a stepwise variable selection, classification and regression trees, threshold-based algorithms, such as the Panelomix algorithm, Naive Bayes and random forest classifiers, among others. The present inventors have found that logistic regression after a stepwise variable selection, classification and regression trees, and threshold-based algorithms achieve similar performance in the context of the present invention, suggesting the importance of the analytes (i.e., biomarkers) used in the methods of the invention, rather than the method used to generate the algorithmic model.

As previously indicated, also another aspect of the invention is a computer-implemented method for carrying out the methods as defined in any of the first and second aspects, in which after the determination of the level of HFABP, said level is given a value and/or a score, and optionally it is computed in a mathematical formula to obtain a computed value; wherein in function of the said level, score and/or computed value, a decision is taken between the options of suffering an LVO and/or being candidate to a reperfusion therapy.

In a particular embodiment, said mathematical formula is the one executed by a particular algorithm.

Herewith disclosed is also a computer-implemented method for carrying out the method for the diagnosis of LVO, or the method for the selection of a subject for a reperfusion therapy, in which after the determination of the level of a FABP such as HFABP, and if determined of the level of one or more of a natriuretic peptide selected from a B-type natriuretic peptide (BNP) and/or atrial natriuretic peptide (ANP); DDi; and GFAP in the isolated sample of the subject, and/or if determined of the one or more clinical parameters of the subject, said level(s) are given a value and/or a score, and optionally are computed in a mathematical formula to obtain a computed value; wherein in function of the said level(s), score(s) and or computed value(s), a decision is taken between the options of suffering an LVO and/or being candidate to a reperfusion therapy, and/or between the options of suffering an ischemic stroke or of any other condition selected from an haemorrhagic stroke, a transient ischemic attack (TIA) and a mimicking stroke.

Throughout the description and claims the word “comprise” and variations of the word, are not intended to exclude other technical features, additives, components, or steps. Furthermore, the word “comprise” encompasses the case of “consisting of”. Additional objects, advantages and features of the invention will become apparent to those skilled in the art upon examination of the description or may be learned by practice of the invention. The following examples are provided by way of illustration, and they are not intended to be limiting of the present invention. Furthermore, the present invention covers all possible combinations of particular and preferred embodiments described herein.

### EXAMPLES

### Example 1

1. Cohorts of Assayed Patients for the Diagnosis of LVO Ischemic Strokes (Abbrev. LVO-IS or LVO)

| | Ambulances- | |
|---|---|---|
| | extra-hospital | ED-hospital |
| | (N = 182) | (N = 197) |
| Sex (female) | 41.2% | 46.7% |
| Age | 72 | 72 |
| | (62.000, | (62.000, |
| | 79.750) | 80.000) |
| Smokers | 19.8% | 17.3% |
| Hypertension | 74.2% | 70.1% |
| Dyslipidemia | 44.5% | 50.8% |
| Diabetes | 30.8% | 23.9% |
| Atrial fibrillation | 19.8% | 30.5% |
| NIHSS (median) | 7 (3-15) | 7 (2-16) |
| ASPECTS (median) | 10 (9-10) | 10 (9-10) |
| Blood samples obtention | 2.167 | 2.583 |
| (hours since stroke onset/median) | (1.083, 5.250) | (1.392, 3.767) |
| Type of disease among stroke | | |
| suspicions: | | |
| Ischemic stroke | 65.9% | 74.1% |
| hemorrhagic stroke | 17.0% | 13.2% |
| mimics | 17.0% | 12.7% |
| LVO (strict definition) | 34.1% | 39.1% |
| Ischemic Stroke Etiology | | |
| atherothrombotic | 23.3% | 11.6% |
| cardioembolic | 34.2% | 39.7% |
| undetermined | 30.0% | 16.4% |
| lacunar | 10.0% | 28.1% |
| tPA therapy | 18.1% | 36.6% |
| Thrombectomy | 30.2% | 28.9% |
| mRS at 3 m (median) | 3 (2-5) | 2 (1-4) |

### 2. Methods for the Two Cohorts and Statistics

### ED-Hospital (N=197)

Patients with suspected stroke (<6 hours) were enrolled when arrived to the emergency department (ED). Blood samples were collected at hospital admission and HFABP, NT-proBNP, D-dimer, GFAP and RBP-4 were measured by immunoassays and POCT.

### Ambulances-Extra-Hospital (N=182)

Patients with suspected stroke (<6 hours) were enrolled in a network of ambulances. Blood samples were collected at patients' home or at the ambulance and HFABP, NT-proBNP, D-dimer, GFAP were measured by immunoassays and POCT.

To explore the markers classification performance and panels feasibility, several combinations of interesting markers were tried using three different approaches: Logistic Regression after a Stepwise variable selection, CART classification tree, Breiman et al. (1984), and Panelomix, a threshold-based algorithm to create panels of biomarkers, Robin et al. (2013) (2.). The output models were validated from the three methods on measurements from another cohort. All analyses were made using R (1.) statistical software, version 4.0.2, using implementations available in Panelomix, Robin et al. (2013), and rpart, Therneau et al. (2019) (3.) libraries.
1. Core Team (2020). R: A language and environment for statistical computing. R Foundation for Statistical Computing, Vienna, Austria. URL https://www.R-project.org/.
2. Xavier Robin, Natacha Turck, Alexandre Hainard, Natalia Tiberti, Frédérique Lisacek, Jean-Charles Sanchez and Markus Müller (2011). Panelomix: A threshold-based algorithm to create panels of biomarkers. Translational Proteomics, 1(1), p. 57-64. DOI: 10.1016/j.trprot.2013.04.003 <http://dx.doi.org/10.1016/j.trprot.2013.04.003>
3. Terry Therneau and Beth Atkinson (2019). rpart: Recursive Partitioning and Regression Trees. R package version 4.1-15. https://CRAN.R-project.org/package=rpart

### 3. Results

3.1. HFABP in Combination with Clinical Parameters and Optionally Other Proteins Provide High Specificities with Accompanying High Sensitivities for LVO and Other Stroke-Related Screening of Patients

Next Tables 1 and 2 show the markers classification performance and panels feasibility of different combinations including the determination of the levels of HFABP in the isolated sample together with data derived from clinical parameters (i.e., blood pressure, age, scores from systematic assessment tools of stroke-related neurologic deficits, and levels of other proteins in the sample). Table 1 includes combinations with the Cincinnati score tool, and Table 2 the combinations with the NIHSS score tool. Sensitivities (SE) and Specificities (SP) are indicated for the combinations allowing:
Diagnosis of LVO ischemic stroke (second column)
Classification or diagnosis of ischemic stroke in relation with other pathologies including strokes (haemorrhagic), mimics and TIAs (third column)
Classification of patients with either an LVO or an haemorrhagic stroke in relation with mimics or non-LVO ischemic strokes

TABLE 1
Combinations of HFABP with data from Cincinnati score tool

| | LVO vs | | | | LVO + | |
|---|---|---|---|---|---|---|
| | Hemorraghic/ | | | | Hemorrhagic | |
| | mimic/non- | | Ischemic vs. | | vs. | |
| | LVO | | Hemorrhagic/ | | non-LVO/ | |
| | ischemic | | mimics/TIAs | | mimics | |
| Panels | SE | SP | SE | SP | SE | SP |
| HFABP + | 20.6% | 100% | 28.6% | 96.9% | 28% | 100% |
| Cincinnati + DBP + | | | | | | |
| age | | | | | | |
| HFABP + | 50% | 93.2% | 14.3% | 100% | 28% | 100% |
| Cincinnati + SBP + | | | | | | |
| age | | | | | | |
| HFABP + | 20.6% | 100% | 14.3% | 100% | 54% | 95.3% |
| Cincinnati + mean | | | | | | |
| BP + age | | | | | | |
| HFABP + BNP | 29.4% | 96.6% | 22.9% | 96.9% | 22% | 95.3% |
| HFABP + BNP + | 41.2% | 100% | 42.9% | 96.9% | 52% | 100% |
| Cincinnati + | | | | | | |
| DBP + age | | | | | | |
| HFABP + DDi | 32.4% | 93.2% | 22.9% | 96.9% | 6% | 97.7% |
| HFABP + DDi + | 44.1% | 100% | 40% | 96.9% | 32% | 100% |
| Cincinnati + | | | | | | |
| DBP + age | | | | | | |
| HFABP + GFAP | 8.8% | 98.3% | 8.6% | 100% | 30% | 95.3% |
| HFABP + GFAP + | 20.6% | 100% | 28.6% | 96.9% | 60% | 95.3% |
| Cincinnati + | | | | | | |
| DBP + age | | | | | | |
| HFABP + | 38.2% | 94.9% | 31.4% | 96.9% | 14% | 97.7% |
| BNP + DDi | | | | | | |
| HFABP + | 52.9% | 100% | 48.6% | 96.9% | 54% | 100% |
| BNP + DDi + | | | | | | |
| Cincinnati + | | | | | | |
| DBP + age | | | | | | |
| HFABP + | 11.8% | 100% | 20% | 100% | 38% | 95.3% |
| BNP + GFAP | | | | | | |
| HFABP + BNP + | 41.2% | 100% | 42.9% | 96.9% | 64% | 95.3% |
| GFAP + | | | | | | |
| Cincinnati + | | | | | | |
| DBP + age | | | | | | |
| HFABP + | 26.5% | 94.9% | 22.9% | 96.9% | 30% | 95.3% |
| GFAP + DDi | | | | | | |
| HFABP + | 44.1% | 100% | 42.9% | 96.9% | 60% | 95.3% |
| GFAP + DDi + | | | | | | |
| Cincinnati + | | | | | | |
| DBP + age | | | | | | |

TABLE 2
Combinations of HFABP with data from NIHSS score tool

| | LVO vs | | | | LVO + | |
|---|---|---|---|---|---|---|
| | Hemorraghic/ | | | | Hemorrhagic | |
| | mimic/ | | Ischemic vs. | | vs. | |
| | non-LVO | | Hemorrhagic/ | | non-LVO/ | |
| | ischemic | | mimics/TIAs | | mimics | |
| Panels | SE | SP | SE | SP | SE | SP |
| HFABP + | 20% | 100% | 28% | 100% | 55.9% | 100% |
| NIHSS + | | | | | | |
| DBP + age | | | | | | |
| HFABP + | 32.5% | 97.4% | 26.2% | 100% | 55.9% | 98.2% |
| NIHSS + | | | | | | |
| SBP + age | | | | | | |
| HFABP + | 50% | 93.4% | 23.8% | 100% | 62.7% | 96.5% |
| NIHSS + | | | | | | |
| mean BP + | | | | | | |
| age | | | | | | |
| HFABP + BNP | 27.5% | 96.1% | 23.8% | 97.3% | 10.2% | 98.2% |
| HFABP + | 35% | 100% | 42.9% | 97.3% | 54.2% | 100% |
| BNP + NIHSS + | | | | | | |
| DBP + age | | | | | | |
| HFABP + DDi | 30% | 93.4% | 23.8% | 97.3% | 6.8% | 98.2% |
| HFABP + DDi + | 37.5% | 100% | 40.5% | 100% | 55.9% | 100% |
| NIHSS + DBP + | | | | | | |
| age | | | | | | |
| HFABP + GFAP | 7.5% | 98.7% | 14.3% | 97.3% | 27.1% | 96.5% |
| HFABP + | 52.5% | 93.4% | 23.8% | 100% | 67.8% | 96.5% |
| GFAP + | | | | | | |
| NIHSS + | | | | | | |
| DBP + age | | | | | | |
| HFABP + | 45% | 92.1% | 33.3% | 97.3% | 13.6% | 98.2% |
| BNP + DDi | | | | | | |
| HFABP + BNP + | 50% | 100% | 52.4% | 97.3% | 54.2% | 100% |
| DDi + NIHSS + | | | | | | |
| DBP + age | | | | | | |
| HFABP + | 12.5% | 100% | 21.4% | 100% | 30.5% | 96.5% |
| BNP + GFAP | | | | | | |
| HFABP + BNP + | 50% | 97.4% | 42.9% | 97.3% | 69.5% | 96.5% |
| GFAP + | | | | | | |
| NIHSS + | | | | | | |
| DBP + age | | | | | | |
| HFABP + | 27.5% | 94.7% | 23.8% | 97.3% | 27.1% | 96.5% |
| GFAP + DDi | | | | | | |
| HFABP + | 60% | 94.7% | 40.5% | 100% | 67.8% | 96.5% |
| GFAP + DDi + | | | | | | |
| NIHSS + | | | | | | |
| DBP + age | | | | | | |

In these tables 1 and 2, when BNP is listed it stands for the determining of the fragment NT-proBNP in the isolated sample. The tables 1 and 2 do not indicate the cut-offs for each of the combinations, since as previously disclosed, the values may vary depending on the type of population, race or even region due to a genetic background. But the main goal, which is demonstrated with the data in the Tables is that HFABP, in combination with one or more clinical parameters and/or with the score obtained from several systematic assessment tools of stroke-related neurologic deficits, allowed a correct diagnosis and classification of patients with specificities near 100%, which is of most relevance in the pathological scenarios under evaluation, and accompanied advantageously with high sensitivities.

Anyway, next Table 3 shows for some of the combinations of the invention (some of them also listed in Tables 1 and 2), the particular cut-offs of the levels of the proteins (including HFABP), of the values of scores from Cincinnati or NIHSS tools, of age, blood pressure (mean, systolic or diastolic) and optionally cut-offs of the levels of other proteins in the isolated sample, giving such high specificities and sensitivities.

As a general rule, inventors have realized that cut-offs for the levels of HFABP selected within the range from 1.25 ng/ml to 6 ng/ml allowed these very good classifications for LVO diagnosis.

TABLE 3
Cut-offs of particular combinations with high specificities
and sensitivities for LVO diagnosis

HFABP + NIHSS + mean BP + age
HFABP > 3.55 ng/ml, NIHSS > 5.5, Age > 69.5, mean BP > 85.67
93.4% specificity & 50% sensitivity
HFABP_+ NTproBNP + Cincinnati + Age + DBP
HFABP > 3.55 ng/ml, NTproBNP > 522.85 pg/ml Cincinnati > 2.5,
Age > 79.5, DBP > 69
100% specificity & 41.2 sensitivity
HFABP + d-dimer + Cincinnati + Age + DBP
HFABP > 4.75 ng/ml, DDimer > 882.729 ng/ml, Cincinnati > 2.5,
Age > 79.5, DBP > 66
100% specificity & 44.1% sensitivity

### 3.2. Prediction of Type of Response to Treatment (Thrombectomy)

As indicated, inventors have also realized that when the levels of HFABP in the isolated sample indicate that the subject is suffering and LVO, and said levels are highly elevated in relation to the cut-off or reference for diagnosing LVO, the subject is likely to show a poor response to therapy in terms of the dependency degree after the stroke episode.

Next sections disclose the use of HFABP levels in the isolated sample of patients as a marker to predict the response to the usually applied therapy:

(A) HFABP and age are independent predictors of response to therapy measured as functional outcome 3 months after stroke

Logistic regression was performed to measure the effects of H-FABP on patient dependency 3 months after stroke. To avoid confusion effects the model was adjusted by the state of dependency of the patient at admission and age. Results show a statistically significant model X2(3)=13.75, 0.003 with a moderate fit as shown by a Naglekerke R2 of 0.129, but it accurately classifies 66.4% of cases. According to the fit, a four-unit increase in HFABP (ng/ml) measured at POCT increases odds ratio of dependency at three months by 2.006 (p-value=0.040), hence duplicating the risk of an mRS score higher than 2.

(B) HFABP, GFAP and age are independent predictors of response to therapy measured as functional outcome 3 months after stroke

Logistic regression was performed to measure the effects of HFABP and GFAP on patient dependency 3 months after stroke. To avoid confusion effects the model was adjusted by the state of dependency of the patient at admission and age. Results show a statistically significant model X2(4)=20.76, <0.001 with a moderate fit as shown by a Naglekerke R2 of 0.190, but it accurately classifies 72.3% of cases. According to the fit, a four-unit increase in H-FABP ng/ml measured at POCT increases odds ratio of dependency at three months by 2.006 (p-value=0.041), hence duplicating the risk of an mRS score higher than 2. On the other hand, a rise of 700 units on GFAP pg/ml while keeping the other variables still produces a similar shift in risk, getting an odds ratio of 2.014 (p-value=0.034).

3.3. Determination of HFABP in Combination with Other Markers Allows Improving the Percentage of Independence of a Patient after an LVO Ischemic Stroke. Taking Key Decisions and Fast Treatment Two Hours Before.

The results on the blood biomarkers of this section come from previous findings in studies performed with samples of patients with stroke suspicion upon arrival at the emergency room. The results have been replicated in real life with samples obtained in ambulances (BIO-FAST study, Biomarkers for Initiating Onsite and Faster Ambulance Stroke Therapies, ClinicalTrials.gov identifier: NCT04612218).

Inventors have realized, by analysing the moment in which these patients received thrombectomy and the moment in which the biomarker information was already available in the ambulance, that the decision to have gone directly to a thrombectomy center could have been made on average two hours earlier.

But inventors went one step forward in determining which was the clinical benefit of starting treatment in a group of patients two hours earlier. This was performed using some calculator-type predictive models such as the one developed in one of the first and largest trials of thrombectomy in stroke (MR CLEAN) called MR PREDICT (https://mrpredicts.shinyapps.io/RRRR_1/), which includes clinical data and image of the patient, as well as at the time of initiation of treatment with thrombectomy. With these calculators inventors were able to do simulations seeing what would have happened if patients with LVO had been treated before, by deriving them directly to the thrombectomy center from the ambulance. According to obtained data, the absolute improvement in % of independent patients (mRS 0-2) would have been 15% using the selected biomarkers (i.e. combinations with HFABP).

It is known that the absolute benefit obtained with thrombectomy is also around 15% of gain in independent patients three months after receiving the treatment. Thus, for example, in some of the large thrombectomy studies, such as the study carried out in Catalonia (REVASCAT), independence went from 28.2% at 3 months in the untreated group to 43.7% in the mechanical thrombectomy group (absolute benefit of the 15.5%); in ESCAPE from 29.3% to 53% (absolute benefit 23.7%); in MR CLEAN from 19.1% to 36.6% (absolute benefit of 17.5%) and in the recent Brazilian study (RESILIENT) from 20% to 35% (absolute benefit of 15%).

It is, therefore, an enormous clinical benefit to have markers that can allow the taking of a fast decision to go to the adequate centre and adequate intervention rooms (e.g., angio-suites). HFABP, as an example of a FABP family member, allows it and reinforces and encourages the application of this novel clinical strategy (i.e., detect LVO patients and send them to an angio-suite as soon as possible).

### Example 2

A Rapid Point of Care Blood Test to Diagnose LVO Patients and Refer them Directly to the Thrombectomy Center Adds Prognostic Information on the Success of Endovas Reperfusion Therapies.

A rapid blood test measuring FABP (i.e., HFABP)/NT-proBNP used in the field among stroke suspicion patients identifies those with LVO with precise specificity/sensitivity and might allow referring them to thrombectomy centers or even attempting direct shift to the angio-suite (BIOFAST, NCT04612218). On top of its diagnostic accuracy, the prognostic value of LVO-check was evaluated among patients with confirmed LVO (n=320) that receive thrombectomy.

### Methods:

Consecutive patients with confirmed LVO were tested with the rapid test for both HFABP/NT-proBNP in a blood sample obtained before thrombectomy was performed. Functional outcome at 3 months was the main endpoint (independent=mRS 0-2 vs. dependent=mRS 3-6) and also mortality at hospital discharge was evaluated.

### Results:

320 patients were included. Regarding functional outcome (n=45 missing data) after thrombectomy 142 (51.6%) remained independent and 133 (48.4%) were dependent at 3 months. Clinical factors significantly associated with poor outcome were age, baseline NIHSS, diabetes and chronic kidney disease. Both HFABP and NT-proBNP were elevated among dependent patients (p<0.001). In the logistic regression [OR (2.5%-97.5%)] baseline NIHSS 1.11 per-point (1.07-1.16; p<0.001), FABP 1.17 per-unit (1.05-1.32; p=0.008) and diabetes mellitus 1.65 (0.94-2.89; p=0.079) were the main independent predictors of outcome. Using Panelomix software optimal cutoffs were identified for age, NIHSS score and FABP to predict poor outcome with excellent specificity (specificity=99.30% and sensitivity=21.10%).

Regarding mortality (n=2 missing data), 289 (90.9%) patients were alive at discharge and 29 (9.1%) patients died at the hospital. Both HFABP and NT-proBNP were significantly elevated among death patients (p<0.001).

Using Panelomix software optimal cutoffs were identified for age, NIHSS score and FABP to predict mortality with excellent specificity (specificity=99.70% and sensitivity=25.90%). Optimal cutoffs were also identified for age, NIHSS score, systolic blood pressure and FABP to predict mortality with excellent sensitivity (specificity=50.90% and sensitivity=100%).

### Conclusions:

Apart from its diagnostic LVO capabilities FABP and NT-proBNP might predict outcome among those patients transferred to thrombectomy centers that get endovascular therapies. This might aid in future decisions about personalizing endovascular therapies and to add neuroprotective strategies in those with predicted poor outcome in spite of successful reperfusion.

### CITATION LIST

### Patent Literature

WO2020229691
WO2018229144

### Non Patent Literature

Gandhi C D, Al Mufti F, Singh iP, et al. Neuroendovascular management of emergent large vessel occlusion: update on the technical aspects and standards of practice by the Standards and Guidelines Committee of the Society of Neurointerventional Surgery. J Neurointerv Surg 2018; 10:315-20.
Lakomkin N, Dhamoon M, Carroll K, et al. Prevalence of large vessel occlusion in patients presenting with acute ischemic stroke: a 10-year systematic review of the literature. J Neurointerv Surg 2019; 11:241-5.
Waqas M, et al. Effect of definition and methods on estimates of prevalence of large vessel occlusion in acute ischemic stroke: a systematic review and meta-analysis. J Neurointerv Surg. 2020 March; 12(3):260-265).
Vanacker P, Heldner M R, Amiguet M, et al. Prediction of large vessel occlusions in acute stroke: National institute of Health Stroke Scale is hard to beat. Crit Care Med 2016; 44:e336-43.
Rai A T et al. (2017). A population-based incidence of acute large vessel occlusions and thrombectomy eligible patients indicates significant potential for growth of endovascular stroke therapy in the USA. J Neurointerv Surg. 9:722-6.
Crowe R P, Myers J B, Fernandez A R, Bourn S, McMullan J T. The Cincinnati Prehospital Stroke Scale Compared to Stroke Severity Tools for Large Vessel Occlusion Stroke Prediction. Prehosp Emerg Care. 2020 Feb. 25:1-9.
So-Young et al. (2012). Plasma heart type fatty acid binding protein level in acute ischemic stroke: comparative analysis with plasma S100B level for diagnosis of stroke and prediction of long-term clinical outcome. Clinical Neurology and Neurosurgery 115, pp.: 405-410.
Noorian A R, Sanossian N, Shkirkova K, Liebeskind D S, Eckstein M, Stratton S J, Pratt F D, Conwit R, Chatfield F, Sharma L K, Restrepo L, Valdes-Sueiras M, Kim, Tenser M, Starkman S, Saver J L; FAST-MAG Trial Investigators and Coordinators. Los Angeles Motor Scale to Identify Large Vessel Occlusion: Prehospital Validation and Comparison With Other Screens. Stroke. 2018 March; 49(3):565-572
Gropen T I, Boehme A, Martin-Schild S, Albright K, Samai A, Pishanidar S, Janjua N, Brandler E S, Levine S R. Derivation and Validation of the Emergency Medical Stroke Assessment and Comparison of Large Vessel Occlusion Scales. J Stroke Cerebrovasc Dis. 2018 March; 27(3):806-815.
Core Team (2020). R: A language and environment for statistical computing. R Foundation for Statistical Computing, Vienna, Austria. URL https://www.R-project.org/.
Xavier Robin, Natacha Turck, Alexandre Hainard, Natalia Tiberti, Frédérique Lisacek, Jean-Charles Sanchez and Markus Müller (2011). Panelomix: A threshold-based algorithm to create panels of biomarkers. Translational Proteomics, 1(1), p. 57-64. DOI: 10.1016/j.trprot.2013.04.003 <http://dx.doi.org/10.1016/j.trprot.2013.04.003>
Terry Therneau and Beth Atkinson (2019). rpart: Recursive Partitioning and Regression Trees. R package version 4.1-15. https://CRAN.R-project.org/package=rpart.

What is claimed is:

1-24. (canceled)

25. A method for treating large vessel occlusion (LVO) in a subject, said method comprising:
a) obtaining an isolated sample from the subject;
b) determining the level of a fatty acid binding protein (FABP) in the isolated sample, and comparing said level of FABP with a reference value or range;
c) diagnosing the subject with LVO:
when the reference value or range is of a subject suffering from LVO and the level of FABP in the isolated sample is equal or within said value or range; or
when the reference value is a cut-off value discriminating between LVO from any other condition selected from one or more of a non-LVO ischemic stroke, an haemorrhagic stroke, a mimic, and a health subject, and level of FABP in the isolated sample is higher than the cut-off; or
when the reference value is of a subject not suffering from LVO, and the level of FABP in the isolated sample is higher than the reference value; and
d) treating the subject diagnosed with LVO with reperfusion therapy.

26. The method according to claim 25, wherein the reperfusion therapy is thrombectomy.

27. The method according to claim 25, wherein the reperfusion therapy is thrombectomy in combination with a previous administration of a thrombolytic and/or fibrinolytic and/or neuroprotective drug.

28. The method according to claim 25, wherein the fatty acid binding protein is heart-type fatty acid binding protein (HFABP).

29. The method according to claim 25, further comprising determining one or more clinical parameters of the subject.

30. The method according to claim 29, wherein the clinical parameters of the subject are selected from the group consisting of blood pressure, including systolic blood pressure (SBP) and/or diastolic blood pressure (DBP), mean blood pressure (mean BP), glycemia, age, scores from systematic assessment tools of stroke-related neurologic deficits, time from onset of symptoms, gender, and combinations thereof.

31. The method according to claim 30, wherein the score from systematic assessment tools of stroke-related neurologic deficits is selected from National Institutes of Health Stroke Scale (NIHSS) score, the Rapid Arterial occlusion Evaluation (RACE) scale for stroke, the Cincinnati Prehospital Stroke Scale Compared to Stroke Severity Tools for Large Vessel Occlusion Stroke Prediction (Cincinnati-score or CPSS), Los Angeles Motor Scale (LAMS), Vision-Aphasia-Neglect (VAN), Field Assessment Stroke Triage for Emergency Destination (FAST-ED), or the modified Rankin Scale or Score (mRS).

32. The method according to claim 25, further comprising determining the level of one or more of a natriuretic peptide, d-dimer (DDi), and glial fibrillary acid protein (GFAP), in the isolated sample of the subject.

33. The method according to claim 25, further comprising determining the level of N-terminal fragment of B-type natriuretic peptide (NTproBNP) and d-dimer (DDi), in the isolated sample of the subject.

34. The method according to claim 25, further comprising determining in the isolated sample of the subject the level of one or more of a natriuretic peptide, d-dimer (DDi), and glial fibrillary acid protein (GFAP), and/or determining one or more clinical parameters of the subject, according to one of the following combinations:
(a) HFABP+NIHSS+DBP+age;
(b) HFABP+NT-proBNP+NIHSS+DBP+age;
(c) HFABP+DDi+NIHSS+DBP+age;
(d) HFABP+GFAP+NIHSS+DBP+age
(e) HFABP+NT-proBNP+DDi+NIHSS+DBP+age;
(f) HFABP+NT-proBNP+GFAP+NIHSS+DBP+age;
(g) HFABP+GFAP+DDi+NIHSS+DBP+age
(h) HFABP+NIHSS+SBP+age;
(i) HFABP+NIHSS+mean BP+age;
(j) HFABP+NT-proBNP+NIHSS+mean BP+age
(k) HFABP+Cincinnati+DBP+age;
(l) HFABP+Cincinnati+SBP+age;
(m) HFABP+Cincinnati+mean BP+age;
(n) HFABP+NT-proBNP+Cincinnati+DBP+age;
(o) HFABP+DDi+Cincinnati+DBP+age;
(p) HFABP+GFAP+Cincinnati+DBP+age;
(q) HFABP+NT-proBNP+DDi+Cincinnati+DBP+age;
(r) HFABP+NT-proBNP+GFAP+Cincinnati+DBP+age;
(s) HFABP+GFAP+DDi+Cincinnati+DBP+age; or
(t) HFABP+NTproBNP+Cincinnati+mean BP+age,

35. The method according to claim 25, wherein the level of the FABP is determined within the six first hours after a stroke onset.

36. The method according to claim 25, wherein the isolated sample is a bio fluid.

37. The method according to claim 25, further comprising:
(i) comparing the level of the FABP in the isolated sample with a cut-off value stratifying the subject diagnosed with LVO according to either a dependency degree and/or a mortality rate;
(ii) determining that the patient (i) has a prognosis defined by a dependency degree greater than 2 according to modified ranking score (mRS) determined within 1-5 months after stroke onset, and/or (ii) has a prognosis defined by a three-month after onset mortality rate comprised from 25%-35%, when the level of the FABP is higher than the cut-off value; and
(iii) administering neuroprotective drugs to the subject in addition to the reperfusion therapy.

38. The method according to claim 25, further comprising shifting the subject diagnosed with LVO to a centre for thrombectomy.

39. The method according to claim 38, wherein when the level of FABP in the isolated sample is higher than a reference value that allows discriminating with a specificity of 100% between a subject suffering either an LVO or a haemorrhagic stroke from a subject suffering either a non-LVO ischemic stroke or a mimic stroke, the subject is directly transferred to an angio-suite in the centre for thrombectomy and optionally treated with a thrombolytic and/or fibrinolytic and/or neuroprotective drug.

40. A kit comprising reagent means for simultaneously detecting the levels of a FABP, and of one or more of a natriuretic peptide, DDi, and GFAP.

41. The kit according to claim 40, comprising means for simultaneously detecting the levels of a FABP, a natriuretic peptide, and DDi.

42. The kit according to claim 40, wherein the FABP is HFABP, and/or wherein the natriuretic peptide is NT-proBNP.

43. A method for the treatment of LVO in a subject, said method comprising:
a) obtaining an isolated sample from the subject;
b) determining the level of a FABP in the isolated sample, and comparing said level of FABP with a cut-off value stratifying the patients according to either a dependency degree and/or a mortality rate:
c) determining that the subject (i) has a prognosis defined by a dependency degree greater than 2 according to modified ranking score (mRS) determined within 1-5 months after stroke onset, and/or (ii) has a prognosis defined by a three-month after onset mortality rate comprised from 25%-35%, when the level of the FABP is higher than the cut-off value; and
d) subjecting the subject with a prognosis defined by (i) a dependency degree greater than 2 according to modified ranking score (mRS) determined within 1-5 months after stroke onset, and/or (ii) a three-month after onset mortality rate comprised from 25%-35%, to reperfusion therapy and/or to a therapy with neuroprotective drugs.

44. The method according to claim 43, further comprising determining one or more clinical parameters an/or features of the subject selected from the group consisting of blood pressure, including systolic blood pressure (SBP) and/or diastolic blood pressure (DBP), mean blood pressure (mean BP), glycemia, age, scores from systematic assessment tools of stroke-related neurologic deficits, time from onset of symptoms, gender, and combinations thereof.

45. The method according to claim 43, further comprising determining the level of one or more of a natriuretic peptide, d-dimer (DDi), and glial fibrillary acid protein (GFAP), in the isolated sample of the subject.

46. The method according to claim 43, further comprising determining the level of NTproBNP and DDi, in the isolated sample of the subject.
